SEQUENCE LISTING (6 sequences)

SEQ ID NO: 1 - DNA, 20 bp, Artificial
  hemoxygenase-1 (HO-1) forward
ggtgatggcttccttgtacc

SEQ ID NO: 2 - DNA, 20 bp, Artificial
  hemoxygenase-1 (HO-1) reverse
agtgaggcccataccagaag

SEQ ID NO: 3 - DNA, 25 bp, Artificial
  nuclear factory erythroid 2 (NRF-2) forward
cgagatatacgcaggagaggtaaga

SEQ ID NO: 4 - DNA, 22 bp, Artificial
  nuclear factor erythroid 2 (NRF-2) reverse
gctcgacaatgttctccagctt

SEQ ID NO: 5 - DNA, 22 bp, Artificial
  GAPDH forward
cccatcaccatcttccaggagc

SEQ ID NO: 6 - DNA, 21 bp, Artificial
  GAPDH reverse
ccagtgagcttcccgttcagc

(19) United States
(12) Patent Application Publication
(10) Pub. No.: US 2025/0268971 A1
(43) Pub. Date: Aug. 28, 2025

(54) SAFFRON AS ANTI-INFLAMMATORY AGENT IN TREATING INFLAMMATORY BOWEL DISEASE

(71) Applicant: HOWARD UNIVERSITY, Washington, DC (US)

(72) Inventor: Hassan ASHKTORAB, Washington, DC (US)

(73) Assignee: HOWARD UNIVERSITY, Washington, DC (US)

(21) Appl. No.: 18/037,712

(22) PCT Filed: Nov. 18, 2021
(86) PCT No.: PCT/US21/59830

§ 371 Date: May 18, 2023

Related U.S. Application Data
(60) Provisional application No. 63/115,146, filed on Nov. 18, 2020.

(51) Int. Cl. A61K 36/88; A61K 31/11; A61K 31/7032; A61K 31/7034; A61P 1/04

(52) U.S. Cl. CPC A61K 36/88; A61K 31/11; A61K 31/7032; A61K 31/7034; A61P 1/04

(57) ABSTRACT

A method for treating or mitigating Inflammatory bowel disease (IBD), particularly ulcerative colitis, including administering an effective amount of saffron to a subject in need thereof. A novel treatment in IBD, particularly ulcerative colitis, is thus provided.

### REFERENCE TO RELATED APPLICATIONS

This is a National Stage of PCT/US2021/059830 filed Nov. 18, 2021, which claims the benefit of U.S. Provisional Patent Application No. 63/115,146, filed Nov. 18, 2020, the entire disclosures of all prior applications are incorporated herein in their entirety by reference.

### SEQUENCE LISTING

The instant application contains a Sequence Listing which has been filed electronically in ASCII format and is hereby incorporated by reference in its entirety. Said ASCII copy, created on Nov. 10, 2023, is named A287644SQ_ST25 and 1.45 kb in size.

### TECHNICAL FIELD

Methods pertain to treatment or mitigation of inflammatory bowel disease (IBD), particularly ulcerative colitis (UC). Particularly, methods comprise administering saffron as an anti-inflammatory agent in treatment or mitigation of IBD, particularly ulcerative colitis. Also, an agent for treatment or mitigation of IBD contains saffron as an active ingredient.

### BACKGROUND

Inflammatory bowel disease (IBD), which includes Crohn's disease (CD) and ulcerative colitis (UC), is a chronic inflammatory disorder of the gastrointestinal tract. IBD has no cure and there has been an increasing incidence and prevalence in the United States and throughout the world (Molodecky, N. A. et al. “Increasing incidence and prevalence of the inflammatory bowel diseases with time, based on systematic review”, Gastroenterology 2012; 142:46-54.e42; quiz e30; Malhotra, A. et al. “All-cause hospitalizations for inflammatory bowel diseases: Can the reason for admission provide information on inpatient resource use? A study from a large veteran affairs hospital”, Mil Med Res 2016; 3:28). Moreover, IBD is associated with an increased longitudinal risk of colorectal cancer. IBD patients on certain immunosuppressive therapies can have increased risk for non-Hodgkin lymphoma, and skin cancers (Kelsen, J. et al. “Inflammatory bowel disease: the difference between children and adults”, Inflamm Bowel Dis 2008;14 Suppl 2: S9-11; Travis, S. “Is IBD different in the elderly?”, Inflamm Bowel Dis 2008;14 Suppl 2: S12-3; Charpentier, C. et al. “Natural history of elderly-onset inflammatory bowel disease: a population-based cohort study”, Gut 2014; 63:423-32). Generally, anti-inflammatory and immunosuppressive drugs are used to treat IBD yet there remains unmet needs for new and safe therapies since many patients lose response, experience drug-induce adverse events or intolerances (Cho, E. J. et al. “Anti-inflammatory effects of methanol extract of Patrinia scabiosaefolia in mice with ulcerative colitis”, J Ethnopharmacol 2011; 136:428-3; Sergent, T. et al. “Anti-inflammatory effects of dietary phenolic compounds in an in vitro model of inflamed human intestinal epithelium”, Chem Biol Interact 2010; 188:659-67).

Many patients with IBD turn to complementary/alternative medicine that include traditional plant-based remedies (Cho E. J., et al. Supra) without medical or professional guidance. Natural products, e.g. those derived from plants and herbs, are increasingly used by IBD patients and reported to have some efficacy in experimental models and small clinical trials. Wide variety of mechanism for their benefits have been reported that include: (1) maintenance of the intestinal epithelial barrier or integrity, (2) regulation of macrophage activation, (3) modulation of innate and adaptive immune response, and (4) inhibition of tumor necrosis factor-alpha (TNF-α) activity (Triantafillidis, J. K. et al. “Favorable results from the use of herbal and plant products in inflammatory bowel disease: evidence from experimental animal studies”, Annals of gastroenterology 2016; 29:268-281; Debnath, T. et al. “Natural products as a source of anti-inflammatory agents associated with inflammatory bowel disease”, Molecules 2013; 18:7253-70). There are also limited controlled data indicating the efficacy of alternative herbal agents, such as aloe vera gel, wheat grass juice, Boswellia serrata, and bovine colostrum enemas in the management of patients with UC (Langmead, L. et al. “Review article: complementary and alternative therapies for inflammatory bowel disease”, Aliment Pharmacol Ther 2006; 23:341-9). However, this area is hampered by lack of detailed mechanistic studies and there is also lack of proper studies that assess dosing carefully.

A perennial stemless herb Crocus sativus L. (Iridaceae), commonly known as saffron, is widely cultivated worldwide, especially in Iran, India, Greece, Morocco, Spain, Italy and China. Saffron has several biological activities and is used in folk medicine (Abdullaev, F. I. et al. “Biomedical properties of saffron and its potential use in cancer therapy and chemoprevention trials. Cancer Detect Prev 2004; 28:426-32). Stigmas of the flower (saffron) contain bitter principles (Picrocrocin), volatile agents (Safranal), dye materials (Crocetin and its glycoside Crocin) anthocyanin, carotene, and lycopene (Abdullaev, F. I. et al. supra). These constituents have been reported to have various pharmacological effects such as anti-depressant and anti-cancer activities (Siddiqui, M. J. et al. “Saffron (Crocus sativus L.): As an Antidepressant”, J Pharm Bioallied Sci 2018; 10:173-180; Festuccia, C. et al. “Crocetin and crocin from saffron in cancer chemotherapy and chemoprevention”, Anticancer Agents Med Chem 2018; Ashktorab, H. et al. “Saffron: The Golden Spice with Therapeutic Properties on Digestive Diseases”, Nutrients 2019; 11) as well as enhancing learning and memory effects (Pitsikas, N. et al. “Effects of the active constituents of Crocus sativus L. crocins and their combination with memantine on recognition memory in rats”, Behav Pharmacol 2018; 29:400-412). With regard to carcinogenesis, many in vitro studies report that extracts of saffron and certain components of the herb are able to inhibit the growth of several types of human cancer cells (Bakshi, H. et al. “DNA fragmentation and cell cycle arrest: a hallmark of apoptosis induced by crocin from kashmiri saffron in a human pancreatic cancer cell line”, Asian Pac J Cancer Prev 2010; 11:675-9; Aung, H. H. et al. “Crocin from Crocus sativus possesses significant anti-proliferation effects on human colorectal cancer cells”, Exp Oncol 2007; 29:175-80; Noureini, S. K. et al. “Antiproliferative effects of crocin in HepG2 cells by telomerase inhibition and hTERT down-regulation”, Asian Pac J Cancer Prev 2012; 13:2305-9). However, so far there have been very few in vivo studies conducted to demonstrate the gastroprotective effects of saffron and its constituents. The effects of saffron and its main constituents on gastrointestinal system need further careful consideration and in-depth assessment. Moreover, whether saffron has effects on intestinal inflammation and changes in gut microbiota as a result of ingestion of saffron and its constituents remain to be explored further, since it is used as dietary constituent and diet which would impact the microbiota.

Ulcerative Colitis (UC) part of IBD is a chronic inflammatory disorder and has a high prevalence in the United States and throughout the world, and is a well-known risk factor of colitis-associated cancer, and there is no cure for it (Rubin, S. J. S. et al. “Mass cytometry reveals systemic and local immune signatures that distinguish inflammatory bowel diseases”, Nat Commun 2019; 10:2686; Ashktorab et al 2020, IBD paper; BMC Gastroenterol. 2020 Jun. 5;20 (1): 170. doi: 10.1186/s12876-020-01279-y. PMID: 32503428). There is high interest in alternative herbal agents, in the management of patients with UC (Langmead and Rampton 2006). Immunosuppressive anti-inflammatory agents are the first line of treatment in UC patients. Such agents have major adverse effects including serious infections, as they are administered continuously to tackle the chronic nature of the disease leading to a weakening in immune defenses.

Therefore, complementary treatments that may be provided by safe herbal substance may be considered as a potential strategy to alleviate the inflammatory signaling in UC.

### SUMMARY OF THE INVENTION

The present inventor has conducted an extensive research and has discovered that: saffron provides therapeutic and protective effects in patients with IBD, particularly ulcerative colitis, in part via HO-1/GPx2 upregulation, regulatory innate and adaptive immune cells and maintenance of microbiota homeostasis. According to the present inventor's discovery, saffron improved colon gross and histology features and led to better body weight, disease activity index, colon length and histology score. Saffron significantly decreased pro-inflammatory macrophages (M1), while increasing anti-inflammatory (M2) macrophages. Saffron also increased IL10+ dendritic cells (DCs) and overall IL10+ expression on CD45+ live leukocytes as well as CD4+ FOXP3+ regulatory T cells. Immunoblot analysis showed a significant increase in HO-1/GPX2 protein expression. Saffron treatment maintained the gut microbiota homeostasis by counter selecting pro-inflammatory bacterial groups. Saffron improved clinical response for the main UC indicator fecal calprotectin and in Partial Mayo Scores. Pro-inflammatory markers TNFα, INFγ, IL-6, IL-2, and IL-17a decreased while anti-inflammatory IL-10 increased in subjects using saffron . . .

The present inventor believes disclosed subject matter to be the first to describe the potential role of saffron as a potent modulator of both innate and adaptive immune responses in different colitis models as well as in peripheral blood mononuclear cell (PBMC) and in human subjects.

### BRIEF DESCRIPTION OF THE DRAWINGS

FIG. 1: Mouse model strategy. Schematic presentation of dextran sodium sulfate (DSS) and Trinitrobenzenesulfonic Acid (TNBS) induced colitis mice models and analysis on colon tissues. Colitis was induced by administration of the 3% DSS in drinking water in wild type C57BL/6 mice (day 0-11) and at day 2, mice were gavaged daily with either saffron aqueous extract (SFE) or vehicle control until day 10. The samples were analyzed for HE, FACs, and immunoblots.

FIGS. 2A-2H: Improved gross and histopathological characteristics of colonic mucosa in DSS-and TNBS-induced colitis mice model. 2A) Hematoxylin and eosin-stained (H&E) longitudinal sections of the distal colon, histology score, DSS model (n=4), one-way ANOVA p<0.05; 2B) Disease activity index; (DAI) (n=5), t test p<0.01; 2C) Percent reduction in bodyweight in DSS colitis model, two-way ANOVA, p<0.01; 2D) and 2E) Colon length in DSS colitis model, one-way ANOVA, p<0.0001; 2F) Percent reduction in bodyweight in TNBS colitis model, two-way ANOVA, p<0.001; 2G) and 2H) Colon length in TNBS colitis model, one-way ANOVA p<. 01, p<0.0001.

FIGS. 3A-3B: Fecal lipocalin (Lcn-2) was measured by ELISA. 3A) Lcn-2 level in DSS colitis mice treated with saffron/vehicle, t test p<0.01); 3B) Len-2 level in TNBS colitis mice treated with saffron/vehicle, t test, p<. 01.

FIGS. 4A-4D: Saffron treatment (n=8) increases dendritic cell population (CD45Live+CD64-CD11c+F4/80−) and T cells (Cd45Live+CD3+). 4A) Bar graph showing dendritic cell population in comparison to DSS+ vehicle (n=10) treated mice (n=8); 4B) Increased IL10+ expression on dendritic cells, t test, p<0.05; 4C) Increased T cells population (CD45Live+CD3+) DSS+ vehicle (n=10) treated mice (n=7); 4D) Increase in Tregs (Cd45+ Live+CD3+CD4+ FOXP3+CD25+) DSS+ vehicle (n=4) treated mice (n=4).

FIGS. 5A-5D: Saffron (250 μg/ml, 500 μg/ml) and Safranal treatment (500 μM, 1 mM) decreases secretion of 4A) TNFα+, 4B) IFNg+ on CD4+ T cells, and 4C), 4D) TNFα+, IFNg+ on CD8+ T cells in human PBMcs.

FIGS. 6A-6B: 6A) Saffron treatment increases mRNA expression of nuclear factor erythroid 2 (NRF-2) and its downstream HO-1gene, and 6B) upregulate HO-1 and Gpx proteins as compared to DSS+ vehicle treated mice, t test, p<0.05.

FIGS. 7A-7E: Saffron increases the abundance of gut microbial taxa associated with maintenance of microbiome homeostasis. 7A) Microbiota richness in the colonic mucosa in mice gavazed 3% DSS at base line (day 0 and day 11) and saffron at 20 mg (day 0 and day 11) (n=3-5 mice per cohort); 7B) principal coordinate analysis (PCoA) of the un-weighted UniFrac distance matrix of the colonic mucosa-associated microbiota; symbols represent data from individual mice color-coded by the indicated metadata; the ovals represent clustering by treatment groups; 7C) firmicutes/Bacteroidetes ratio in DSS (day 0.579 and day 11, 0.974) and DSS-SFE (day 0, 0.548, day 11, 0.501) show maintenance of microbiome hemostasis in saffron treated animals; 7D) relative abundance of bacterial order in the colonic mucosa; bars represent mice group; labels indicate families with average relative abundances>1% in at least one treatment group; 7E) relative abundance of mucosa-associated bacterial at genus significantly altered in saffron treated animals; data are presented as means±SEM. * P<. 05 DSS vs DSS-saffron gavaged mice.

FIG. 8: Proposed model of saffron and protection of colitis by immune modulation, target anti ROS markers and microbiome.

FIGS. 9A-9C: Colon length improves with saffron treatment 15 mg/kg b.wt and 20 mg/kg b.wt.

FIG. 10: Phases of Clinical Trial.

FIG. 11: C-Reactive Protein (CRP) Pretreatment and Post-treatment with Saffron for P1, P3, and P4.

FIG. 12: Fecal Calprotectin Pretreatment and Post-treatment with Saffron for P1, P3, and P4.

FIG. 13: Partial Mayo Pretreatment and Post-treatment with Saffron for P1, P3, and P4.

FIG. 14: Hamilton Depression Rating Scale Pretreatment and Post-treatment with Saffron for P1, P3, and P4.

FIGS. 15A and 15B: Intracellular cytokine production measured by CyTOF, in response to 4 h stimulation of PBMC with PMA+ ionomycin for 15A) patient 1 (P1) and 15B) Patient 4 (P4).

FIGS. 16A-16D: Saffron improve gut microbiome. 16A) Reduction of gamma proteobacteria (consist of a lot of pathogens and proinflammatory bacteria), Cyanobacteria, Bacteroidetes in Patient-1 treated with 8 weeks saffron compared to baseline (B); 16B) and 16C) Reduction of Firmicutes in P4, P3 post-treatment with saffron compared to baseline; 16D) saffron gut microbiome analysis of all patients (P1, P3, P4) with decrease in Firmicutes and Actinobacteria with Saffron treatment compared to the baseline.

### DETAILED DESCRIPTION OF THE INVENTION

The cause of IBD is currently unknown and it is believed to stem from the complex interaction between host immune, genetic, and environmental factors. Although over 200 genes have been associated with IBD, monozygotic twin studies indicate that non-genetic (environmental) factors are key drivers in disease pathogenesis (Beaugerie, L. et al. “Risk of new or recurrent cancer under immunosuppressive therapy in patients with IBD and previous cancer”, Gut 2014; 63:1416-23; Jostins, L. et al. “Host-microbe interactions have shaped the genetic architecture of inflammatory bowel disease”, Nature 2012; 491:119-24; de Lange, K. M. et al. “Genome-wide association study implicates immune activation of multiple integrin genes in inflammatory bowel disease”, Nat Genet 2017; 49:256-261). Efforts in elucidating these environmental factors have focused on the interplay between the intestinal microbiome (including commensal bacteria, fungi and viruses) and the host immune response. Whether these are cause or effect relationships remain a hot area of investigation. Recently, it has been shown dysbiosis associated with a deficiency in bacterial metabolites (secondary bile acids, SBAs) in UC patients with pouches compared to control familial adenomatous polyposis (FAP) subjects with pouches (Sinha, S. R. et al. “Dysbiosis-Induced Secondary Bile Acid Deficiency Promotes Intestinal Inflammation”, Cell Host Microbe 2020). Moreover, the inventor has conducted treatment with localized SBAs mitigated inflammation in multiple experimental models of colitis/IBD. The effects of saffron and its main constituents in gastrointestinal system have not been explored comprehensively and the inventor has explored the therapeutic role of saffron using colitis mouse models and mechanisms that lead to beneficial effects as well as determine saffron's effect on dysbiosis that is commonly associated with intestinal inflammation.

The disclosed subject matter provides novel treatment in IBD, particularly ulcerative colitis.

One example embodiment of the disclosed subject matter provides an agent for treatment or mitigation of IBD, containing saffran as an active ingredient.

Another example embodiment of the disclosed subject matter provides a method for treating or mitigating IBD, comprising administering an effective amount of saffron to a subject in need thereof.

In one of its aspects, saffron is administered orally.

In one of its aspects, saffron is in the form of saffron extract.

In one of its aspects, the IBD is good for ulcerative colitis.

In one of its aspects, saffron treatment improves gross and histological features.

In one of its aspects, saffron decreases fecal lipocalin, a sensitive marker of intestinal inflammation.

In one of its aspects, saffron treatment alters innate immune cell profiles.

In one of its aspects, saffron and its constituent safranal decrease secretion of pro-inflammatory cytokines on T cells in human PBMCs.

In one of its aspects, saffron treatment induces cytoprotective transcription factors and downstream anti-inflammatory and anti-oxidant proteins.

In one of its aspects, saffron alters the abundance of microbial taxa associated with inflammation.

Saffron alters the abundance of microbial taxa associated with inflammation.

Saffron enrich Firmicutes and/or deplete which confirm the presence of intestinal microbes such as the Erysopelotrichales Order in protection of epithelial cells.

The formulation of saffron and method of administration of saffron can be selected suitably. Saffron has polar and no-polar ingredients that make it unique in its preparation as either solid or liquid prep for the human consumptions. Crocin is the main component of saffron. Crocin a complex natural product which its laboratory total synthesis is very challenging and time consuming. Crocin is a polyene which is coupled with the diesters formed from the disaccharide gentiobiose and the dicarboxylic acid crocetin. While the esterification step is straightforward, the polyene synthesis is very tedious as the alternating C═C double bonds are arranged in a very specific geometric/configurational isomeric forms. Therefore, the possibility of the formation of many unwanted polyene byproducts is inevitable posing purification challenges given that multi-cis/trans isomers have similar polarities for purification. Instead, as shown by Himeno et al. (Agric. Bioi. Chem. 1987, 51, 2395-2400), the purification of crocin from the extraction dried saffron is very reliable and convenient. The process is based on the utilization of reverse-phase HPLC-MS. This approach also allows for the quantification of isolated crocin derivatives fractions based on the absorption coefficients of the desired crocin derivatives using their characteristic wavelength absorption maxima, e.g. at 250.5 nm, 308 nm, and 443 nm. For example, the saffron can be formulated in solid dosage form, such as a capsule, tablet or the like, or can be formulated in a liquid suitable for administration orally. These formulations can be produced by well-known methods.

Solid drugs for internal use as an oral agent are formulated by, for example, mixing the active ingredient with, for example, a vehicle, a binder, a disintegrant, a lubricant, a stabilizer, a dissolution adjuvant, and the like, and formulating according to standard methods. As necessary, coating may be carried out with a coating agent, and coating of two or more layers may be employed.

Liquid drugs for internal use as an oral agent can be produced by, for example, dissolving, suspending, or emulsifying an active ingredient in a generally used diluent (for example, purified water). A liquid drug may include a wetting agent, a suspension agent, an emulsifying agent, a sweetening agent, a flavoring material, an aromatic substance, a preservative, a buffer agent, and the like.

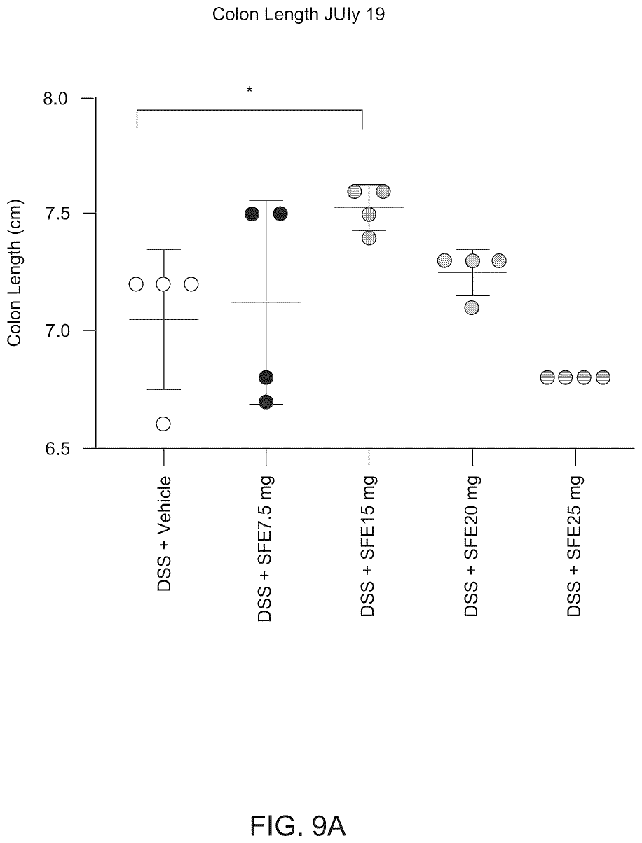

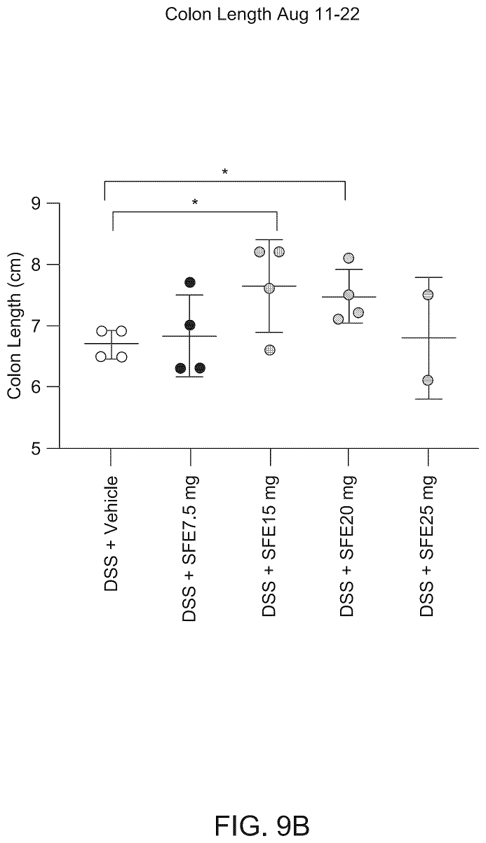

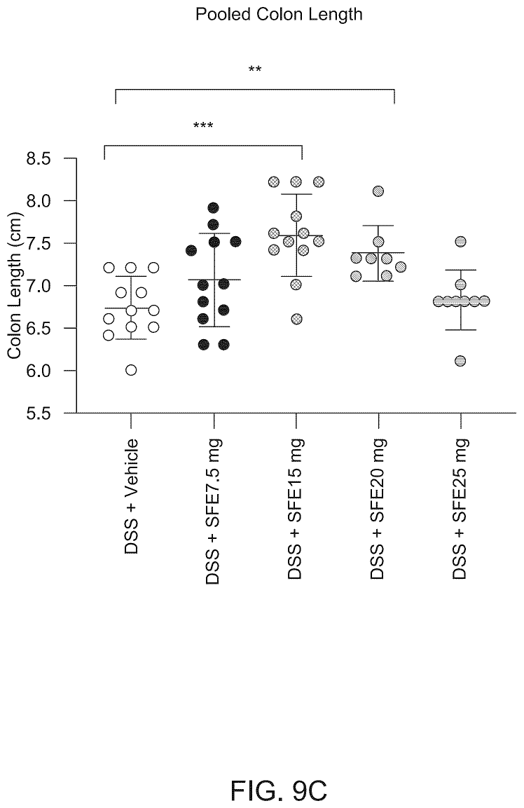

The dose of the saffron to be used is different depending on ages, body weights, symptoms, therapeutic effects, administration method, treatment time, and the like. For example, the dose of the saffron per adult is generally from 5 mg to 100 mg per dose, preferably 10 to 90 mg, or 20 to 80 mg, once or twice per day by oral administration. Dose specification for the saffron depends on the quality of saffron and its ingredients component. Saffron ingredient such as crocin, saffranl, and picrocrocin work synergistically as anti-inflammatory compound. The dose analysis for the concentration of saffron was conducted and the optimum dose was determined. Dose analysis was done in in vivo (mice model) and based on the colon length, M1, M2, and immune markers such as IL-10, TNF-α. FIGS. 9A-9C show that the colon length was increased by treating the mouse with 7.5-20 mg/kg body weight but it decreased in 25 mg/kg body weight. In addition, most of the anti-inflammatory markers such as Il-10, CD3+ T cells, and Dendritic CD11c+CD11b-F480—was less pronounce for 25 mg/kg body weight while significant for <25 mg/kg/body weight. This was the reverse for pro-inflammatory response for saffron dose<25 mg/kg body weight. The dose calculation was based on 7.5-20 mg per kg body weight in mouse and was calculated in human based on body surface area. Needless to say, as mentioned above, the dose to be used varies dependent on various conditions. Therefore, dose lower than the ranges specified above may be sufficient in some cases, and dose higher than the ranges specified above are needed in some cases.

The saffron may be administered in combination with other medicine (for example, well-known agents for treating IBD) for the purposes of: (1) supplementing and/or enhancing therapeutic effect, (2) improving the kinetics, improving absorption, and reducing the dose; and/or (3) eliminating the adverse reaction of the compound.

### Examples

The present invention is explained below in further detail with reference to Examples. However, the scope of the invention is not limited to these Examples.

### Animals

C57BL/6 and Balb/c mice were purchased from Taconic (Hudson, NY) and Jackson Laboratory respectively and housed at the Stanford research animal facility for 1 week until used in experiments. Animals were maintained in accordance with National Institutes of Health guidelines and experiments were approved by Stanford University Institutional Animal Care and Use Committee.

### Saffron Aqueous Extract

Saffron dried stigmas were obtained from Gulf Pearls SPRL (Brussels, Belgium, www.gp-food.com). The quality of saffron was evaluated for batch to batch variation according to ISO3632 standard according to the inventor's previous study (Tabtabaei, S. et al. “Geographical classification of Iranian and Italian saffron sources based on HPLC analysis and UV-Vis spectra of aqueous extracts”, European Food Research and Technology 2019; 245:2435-2446). Saffron dried stigmas were grinded into fine powder and dissolved in water at a concentration of 2 mg/ml in a 15 ml falcon tube on an orbital shaker at room temperature in the dark for one hour. This is called saffron suspension/aqueous extract (SFE). The SFE can be stored for two days at 4° C. The extraction was prepared fresh every second day, for oral administration by gavage.

### Dextran Sodium Sulfate (DSS) Colitis Model

Dextran sodium sulfate (DSS) (36,000-50,000 MW) was purchased from MP Biomedicals (Santa Ana, CA) and dissolved in drinking water to 2.5% (w/v) and given ad libitum to 8-week-old female C57BL/6 beginning on day 0 for 11 days. Body weights were recorded daily. In the control group, the mice were given regular drinking water. Colitis was induced by administration of 3% dextran sodium sulfate (DSS) in drinking water in wild type C57BL/6 mice (day 0-11) and at day 2, mice were gavaged daily with either saffron aqueous extract (SFE) or vehicle control until day 10. To determine effective dose(s), different concentrations of SFE (1, 5, 7.5, 15, 20, 25 mg/kg body weight) were tested. The most effective dose of saffron was further validated and used for different experiments. On day 11, we euthanized the mice and analyzed for gross, clinical, and microscopic evidence of disease that included colon length (severe disease is associated with shorter colon), percent body weight loss, disease activity index (DAI, which also includes stool consistency and blood in feces), and histologic scores (H&E staining). The stool samples (from day 0 and day 11) were snap frozen and kept at −80° C. for Lipocalin fecal levels and bacterial 16S gene sequencing analysis.

### Trinitrobenzenesulfonic Acid (TNBS) Colitis Model

Pre-sensitization TNBS/Ethanol model was used that utilizes skin TNBS exposure a week prior to intra-rectal (i.r.) TNBS to induce a delayed type of hypersensitivity, a colitis model superior to just acute TNBS/Ethanol i.r. exposure without pre-sensitization (Wirtz, S. et al. “Chemically induced mouse models of intestinal inflammation”, Nat Protoc 2007; 2:541-6; Koboziev, I. et al. “Pharmacological intervention studies using mouse models of the inflammatory bowel diseases: translating preclinical data into new drug therapies”, Inflammatory bowel diseases, 2011; 17:1229-1245). Following skin TNBS exposure at day-7, the Balb/c mice received either TNBS or vehicle (50% ethanol) i.r. on day 1 and gavaged daily with SFE or control from day 0 until euthanized on day 4 (Wirtz, S. et al. supra). Colon tissues were harvested for colon length measurement and for H&E staining as in the DSS model above.

### Histology and DAI Score

The colon tissue was fixed in 10% buffered formalin and processed for hematoxylin and eosin (H&E) analysis (Histo-Tec Laboratory, Inc., Hayward, CA). The slides were blindly scored for intestinal inflammation (inflammatory cell infiltrate, intestinal architecture) on a scale of 0-3 as previously described in the literature (Erben, U. et al. “A guide to histomorphological evaluation of intestinal inflammation in mouse models”, Int J Clin Exp Pathol 2014; 7:4557-76). Disease activity index (DAI score) was also calculated by scoring three factors, the body weight loss, stool consistency, and blood in the stool, as described in the literature (Pandurangan, A. K. et al. “Gallic acid attenuates dextran sulfate sodium-induced experimental colitis in BALB/c mice”, Drug design, development and therapy 2015; 9:3923-3934). The loss of the body weight was scored as follows: score 0, no body weight loss; score 1, body weight loss 1%-5%; score 2, body weight loss 6%-10%; score 3, body weight loss 11%-20%; score 4, more than 20% body weight loss. Stool consistency was scored as measured: score 0/1 normal solid pellets; score 2, loose stool with some solidity; score 3, loose stool with some liquidity; score 4, diarrhea. The visible blood in the stool was scored as follows: score 0/1 no blood detected; score 2, slightly bleeding; score 3, moderate bleeding; score 4, gross bleeding. The total sum from body weight, stool consistency and blood in the stool was calculated as the overall DAI score (Pandurangan, A. K. et al. supra).

### Fecal Lipocalin 2 (Lcn-2) ELISA

Fecal samples were collected on the last day of the DSS colitis and TNBS experiment. The fecal samples were reconstituted in PBS containing 0.1% TWEENR-20 at a concentration of 100 mg feces/ml and vortexed for 20 min to yield a homogenous suspension (Chassaing, B. et al. “Fecal lipocalin 2, a sensitive and broadly dynamic non-invasive biomarker for intestinal inflammation”, PLOS One 2012; 7: e44328). Samples were then centrifuged at 12,000 RPM for 10 minutes. Supernatant was collected and stored at −20° C. until analysis. Lipocalin 2 (Lcn-2) levels were quantified using Mouse Lipocalin-2/NGAL ELISA kit according to the manufacturer's recommendation (R&D Systems, Minneapolis, MN). The optical density was measured Spectramax iD3 (Molecular Devices Corp., Sunnyvale, CA, USA) at 450 nm.

### Colon Leukocytes Isolation and Flow Cytometry

Leukocytes were isolated from the colon as described previously (Nguyen, L. P. et al. “Role and species-specific expression of colon T cell homing receptor GPR15 in colitis”, Nat Immunol 2015; 16:207-13). The colons were cleansed in Hank's Balanced Salt Solution (HBSS) containing 2% bovine calf serum (BCS) without Ca²⁺/Mg²⁺. The tissues were first incubated twice with HBSS containing 2% BCS and 2 mM Ethylenediaminetetraacetic acid (EDTA) at 37° C. for 20 minutes. The EDTA was then rinsed with HBSS containing 2% BCS without EDTA. The colon tissues were cut into ˜0.5 cm pieces and incubated twice with collagenase IV (Sigma Aldrich, St Louis, Mo) in 5% BCS Roswell Park Memorial Institute medium (RPMI) at 37° C. (30 minutes each). The cell suspension was then collected and washed. Density gradient centrifugation with 40/80% percoll (GE Healthcare Bio Science AB, Uppsala, Sweden) was used to enrich the leukocytes at the interface. The leukocytes were then washed and stimulated with a mixture of Lipopolysaccharide (LPS) (1 μg/mL)+ phorbol myristate acetate (PMA; 50 ng/ml)+ Ionomycin (1 μg/ml)+ Brefeldin A for 3 hours, before staining for flow cytometry analysis. Two different panels of dye and antibodies were used for staining different immune cells. The first panel includes: Zombie Aqua (Biolegend, San Diego, CA), CD45 Percp5.5 (Biolegend), F4/80 APC-Cy7 (Biolegend), IL10 PE-Cy7 (Biolegend), TNFα-Alexa 488 (Biolegend), CD206 APC (Biolegend), CD11b PETR (Biolegend), CD64 PB (Biolegend), CD11c PE (Biolegend), MHCII (IA/IE) AF700. The second panel includes: Zombie Aqua (Biolegend, San Diego, CA), CD45 Percp5.5 (Biolegend), CD4 Qdots (Biolegend), CD44 PECy7 (Biolegend), CD45RB APC Cy7 (Biolegend), CD25 AF700 (Biolegend), FOXP3 FITC (Biolegend), Tbet PETR (Biolegend), RORGT PE (Biolegend), CD3 APC (Biolegend), CD8a PB (Biolegend). Intracellular staining of cytokines was performed using fixation and permeabilization solutions as per manufacturer recommendations (eBioscience). Data was acquired and analyzed on an LSRII (BD Biosciences) and FlowJo software (FlowJo, LLC), respectively.

### RNA Isolation and Quantitative Real-Time Polymerase Chain Reaction

RNA was extracted from the colon tissue using RNeasy kit (Qiagen, Hilden, Germany). The RNA was converted to complementary DNA using the High-Capacity RNA-to-cDNA kit (Applied Biosystems, Foster City, CA). Quantitative reverse-transcription polymerase chain reaction of the cDNA was conducted on an ABI StepOne Plus real-time instrument (Thermo Fisher Scientific, Waltham, MA) using the following primers: hemoxygenase-1 (HO-1): forward, 5′-GGTGATGGC TTCCTTGTACC-3′ (SEQ ID NO: 1); reverse, 5′-AGTGAGGC CCATACCAGAAG-3′ (SEQ ID NO: 2), nuclear factor erythroid 2 (NRF-2) forward, 5′-CGAGATATACGCAGGAGAGGTAAGA-3′ (SEQ ID NO: 3); reverse, 5′-GCTCGACAATGTTC TCCAGCTT-3′ (SEQ ID NO: 4) (Kim et al., 2012). For each gene, the housekeeping gene GAPDH: forward, 5′-CCCATCAC CATCTTCCAGGAGC-3′ (SEQ ID NO; 5); reverse, 5′-CCAGT GAGCTTCCCGTTCAGC-3′ (SEQ ID NO: 6) (Aoki et al., 2001), was used to normalize. The alteration in gene expression between groups was analyzed using the Pfaffl method (Pfaffl MW. “A new mathematical model for relative quantification in real-time RT-PCR”, Nucleic Acids Res 2001;29: e45).

### Western Blot Analysis

Mouse colon tissues were homogenized in RIPA buffer (Cell Signaling, Danvers, MA) with protease inhibitor cocktail (Sigma, St. Louis, MO). Protein related to NRF-2/HO-1 pathway signaling was detected with antibodies HO-1 (Cell Signaling, Danvers, MA and Gpx-2 antibodies. HO-1 (Cell Signaling, Danvers, MA), Gpx-2 (Thermo Fischer Scientific, USA) and β-actin (Santa Cruz, CA) antibodies were used for western blot.

16S rRNA Sequencing and Data Analysis

The Genome Sequencing Service Center (GSSC) at Stanford University conducted DNA sequencing as previously reported (Sinha, S. R. et al. “Dysbiosis-Induced Secondary Bile Acid Deficiency Promotes Intestinal Inflammation”, Cell Host Microbe 2020; 27:659-670 e5). Details of OTU assembly from reads, abundance estimation of each OTU and taxonomic assignment to be on previous study (Ashktorab, H. et al. supra; Brim, H. et al. “A Microbiomic Analysis in African Americans with Colonic Lesions Reveals Streptococcus sp. VT162 as a Marker of Neoplastic Transformation”, Genes (Basel) 2017;8). OTU abundances were loaded into R (version 3.5.1) using the package phyloseq (McMurdie, P. J. et al. “Phyloseq: an R package for reproducible interactive analysis and graphics of microbiome census data”, PLOS One 2013; 8: e61217). To determine whether the sequencing depth is adequate to fairly represent the OTU richness of the samples, a rarefaction analysis was conducted. To check how the samples from the same experimental condition cluster with each other, the inventor generated a Principal Coordinate Analysis plot using the Jensen-Shannon Divergence distance measure. To detect taxonomic units that are differentially expressed between Saffron treated and DSS group, the inventor did a 2-factor ANOVA with treatment (Saffron vs. Untreated) and days (0 and 11) as the two factors. The analysis was done using the R package DESeq2 (Love, M. I. et al. “Moderated estimation of fold change and dispersion for RNA-seq data with DESeq2”, Genome Biol 2014; 15:550). At each taxonomic rank, the counts of reads falling into OTUs within each taxon were summed up. Those counts were then used to do the ANOVA using DESeq2. Taxons with a false detection ratio (FDR)<0.05 were selected as statistically significant.

### Effect of Saffron and its Constituent Safranal on Human PBMCs T Cells Pro-Inflammatory Cytokine Secretion

A 48 well plate was coated with human anti-CD3 antibody (1 μg/ml) overnight. After washing with 1×PBS, plates were coated with human PBMCs (0.5×10⁶ cells/well), human anti-CD28 (1 μg/ml), β-mercaptoethanol (1 μg/ml), different concentrations of Saffron aqueous extract (250 μg/ml, 500 μg/ml) and other constituents of saffron, Safranal (500 μM, 1 mM) (Sigma Aldrich, USA). The human PBMCS of 0.5×10⁶ cells/well PBMCs were seeded in 48 well microplates in RPMI-1640 (Sigma, USA) supplemented with 10% fetal bovine serum and 1% penicillin/streptomycin at 37° C. and in 5% CO₂) (day 0). At day 3, cells were stimulated with phorbol myristate acetate (PMA; 50 ng/ml)+ Ionomycin (1 μg/ml)+ Brefeldin A (1X) for 4 hrs. and cells were harvested and stained for flow cytometry. The human specific antibodies for T cells (dye; Zombi Aqua, CD45 PerCP 5.5, CD3 BV421, CD4 PETR, CD127-PECy7, CD25 BV605, CD8a AF488 and pro-inflammatory cytokines, like TNF-α AF700, IFNg APC, IL4 PE) were used to stain the PBMC cells.

### Statistical Analysis

Statistical analyses of different sets of experiments was performed using Prism 7 (GraphPad Software, Inc., La Jolla, CA). Two-tailed t-test or Mann-Whitney's test were used in a single variable with two group comparison, one-way ANOVA with Tukey posttest or Kruskal-Wallis test were used in single-variable comparisons with more than two groups, and two-way ANOVA with Bonferroni post-test for multi-variable analyses. Differences with P<. 05 were regarded as statistically significant.

### Results

### Saffron Treatment Improves Gross and Histological Features in DSS and TNBS Colitis Models

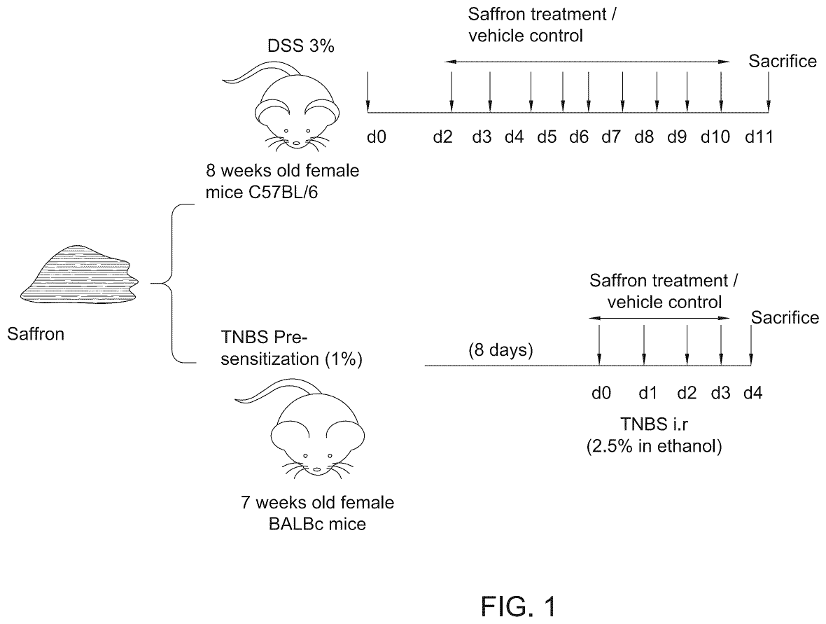

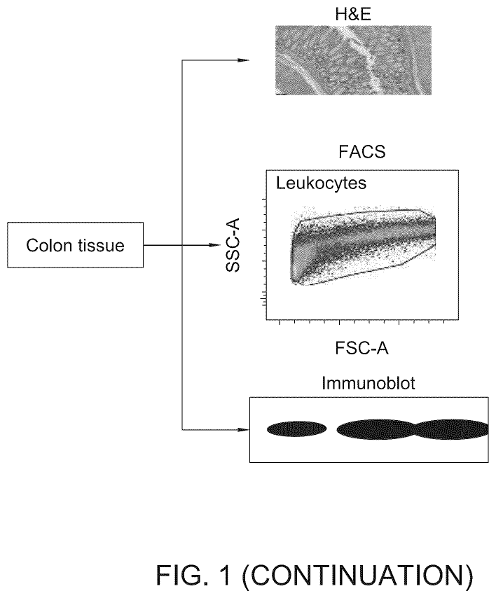

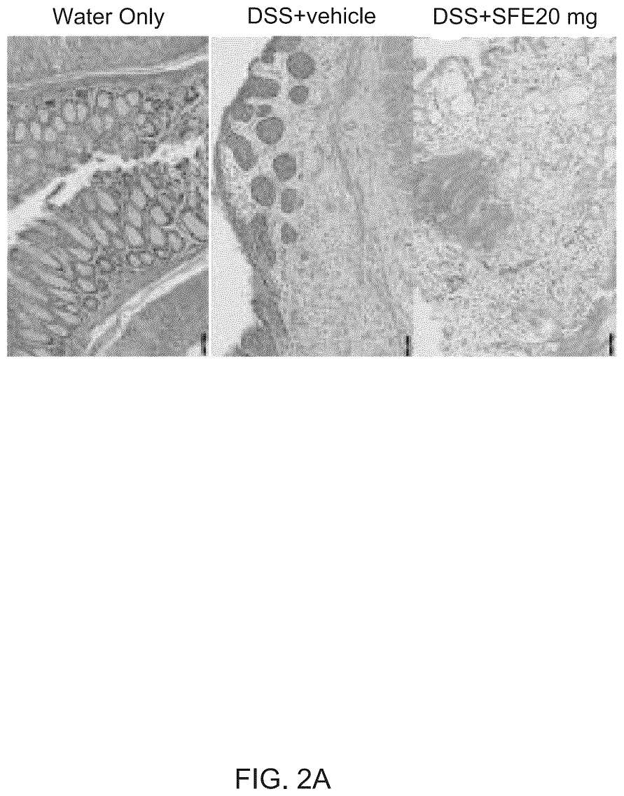

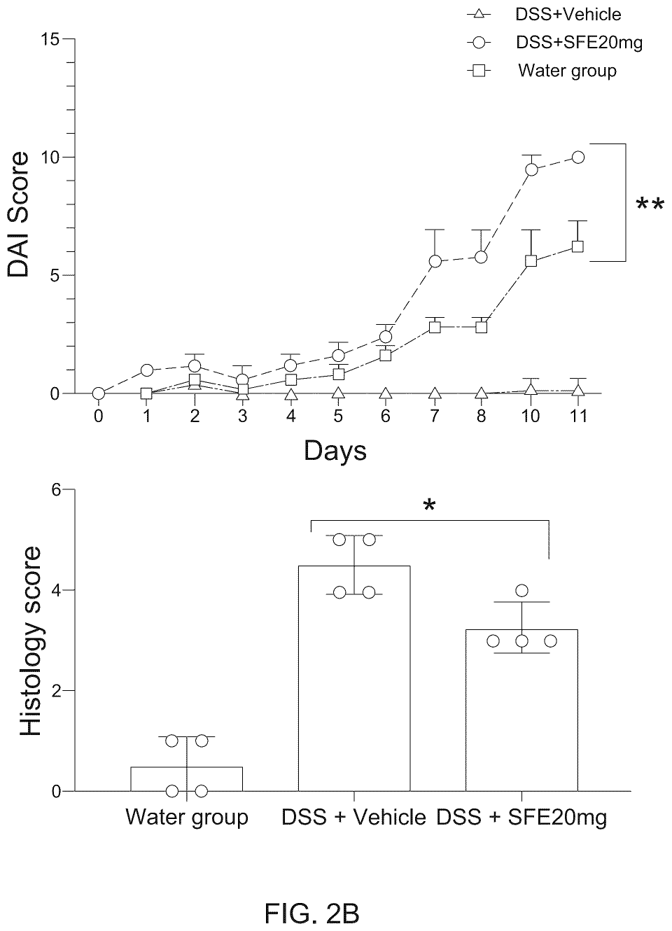

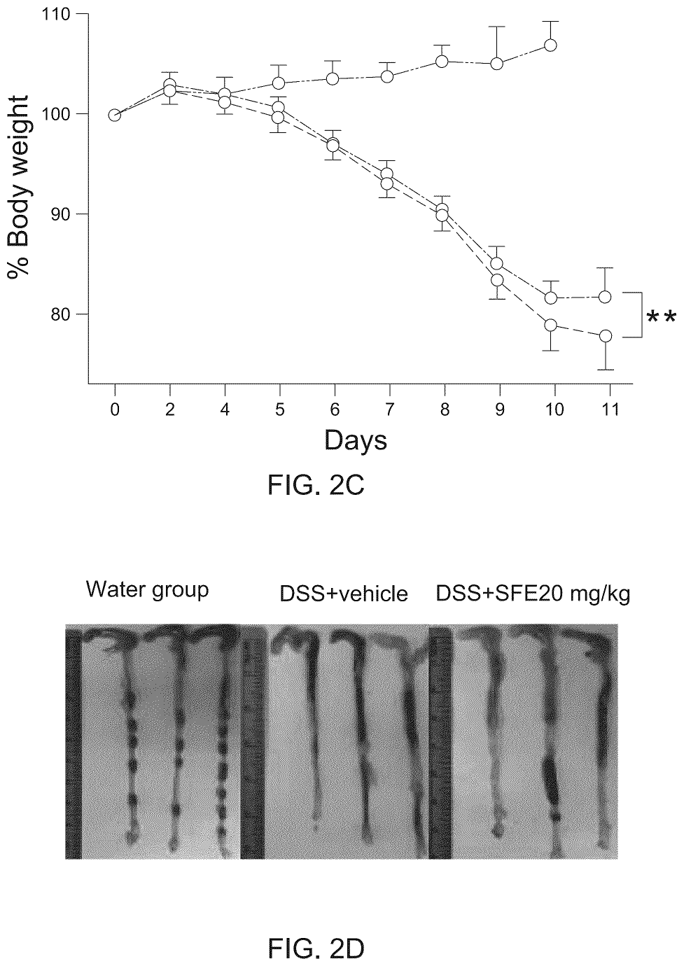

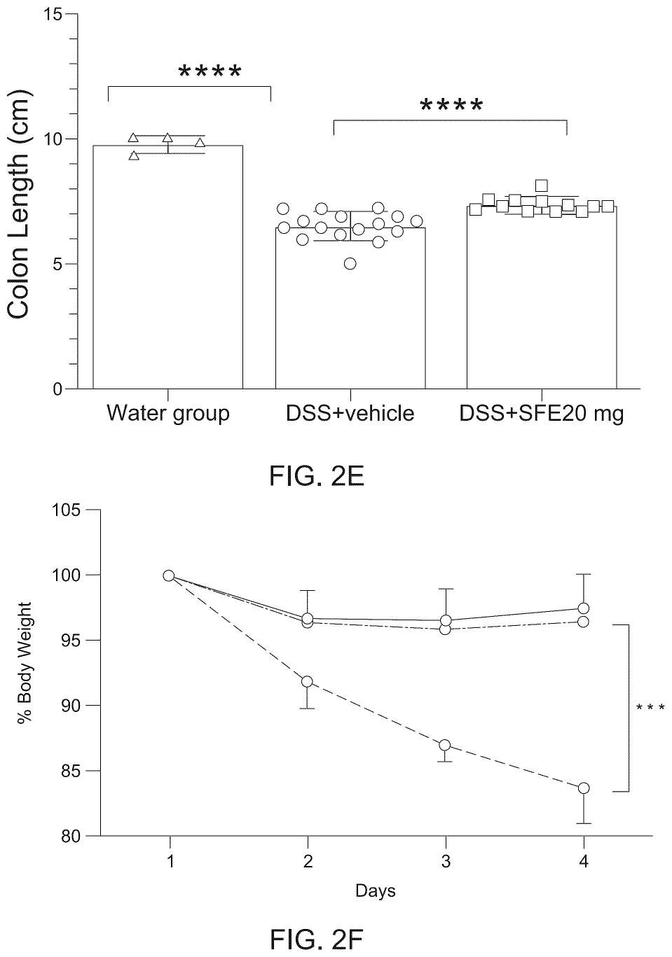

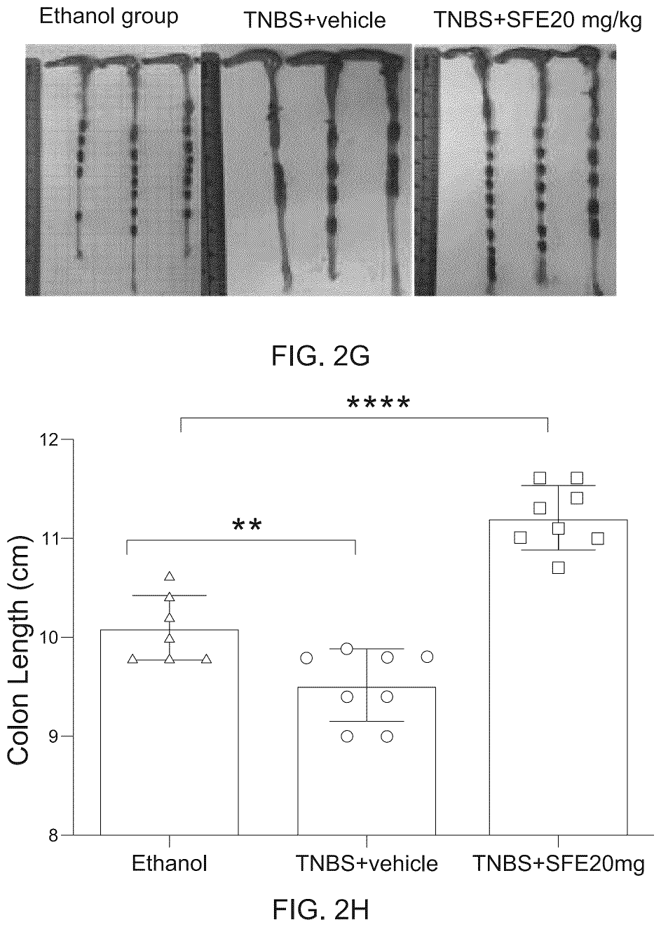

Colitis was induced by administration of the 3% dextran sodium sulfate (DSS) in drinking water in wild type C57BL/6 mice (day 0-11) and at day 2, mice were gavaged daily with either saffron aqueous extract (SFE) or vehicle control until day 10 (FIG. 1). To determine effective dose(s), different concentrations of SFE (1, 5, 7.5, 15, 20, and 25 mg/kg b. wt) were tested. On day 11, the mice were euthanized and analyzed for gross, clinical, and microscopic evidence of disease that included colon length (severe disease is associated with shorter colon), percent body weight loss, disease activity index (DAI, which also includes stool consistency and blood in feces), and histologic scores (H&E staining). It was found that 20 mg/kg body weight SFE dose showed the most protective effect (as shown in FIG. 2) and the severity of colitis in the mice decreased, evident by improved histology score, colon length, body weight and DAI score (FIGS. 2A-D). The protective effect of saffron was also evaluated in TNBS (Trinitrobenzenesulfonic acid) colitis mouse model. Similar to the DSS colitis model, SFE 20 mg/kg body weight treatment decreased colitis which was evident by improved colon length, and body weight (FIGS. 2E-F).

### Saffron Decreases Fecal Lipocalin, a Sensitive Marker of Intestinal Inflammation

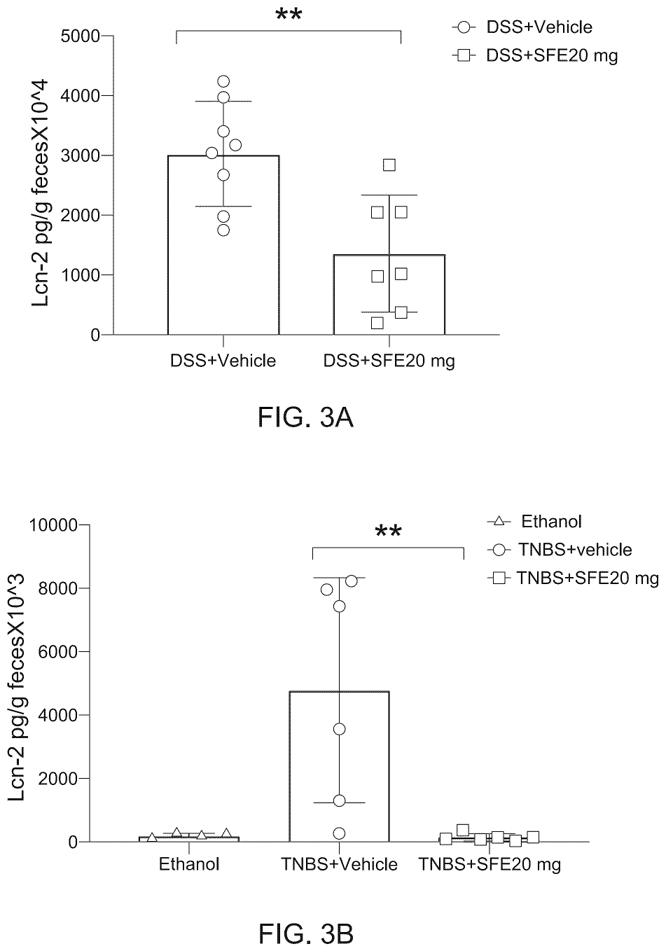

Fecal lipocalin 2 (Lcn-2) is a sensitive and broadly dynamic non-invasive biomarker for intestinal inflammation (Chassaing, B. et al. supra). Fecal samples collected at day 11 from DSS colitis mice and on day 4 from TNBS colitis mice treated with saffron or vehicle were analyzed by Lcn-2 ELISA. SFE 20 mg/kg treatment reduced fecal lipocalin level significantly in both DSS (FIG. 3A) and TNBS colitis mice (FIG. 3B). These results are consistent with clinical and histologic improvements observed in mice treated with SFE (FIG. 2).

### Saffron Treatment Alters Innate Immune Cell Profiles

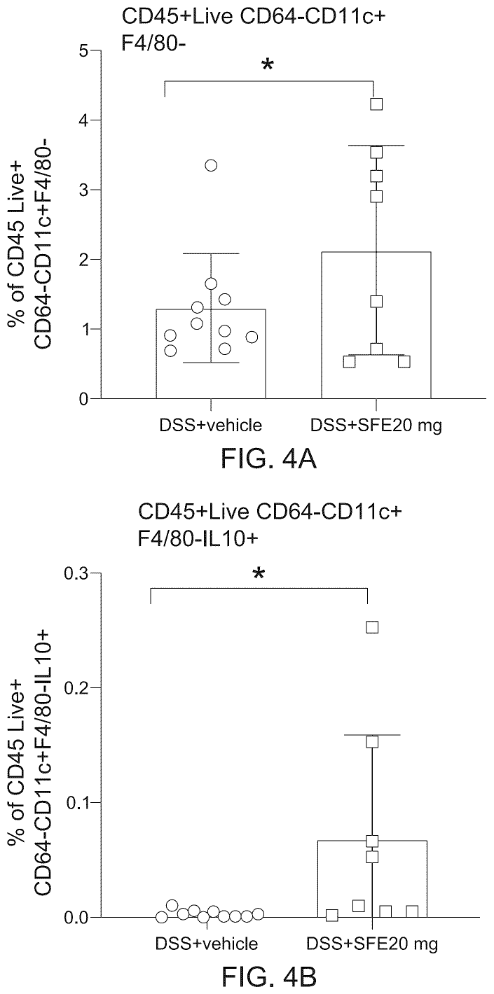

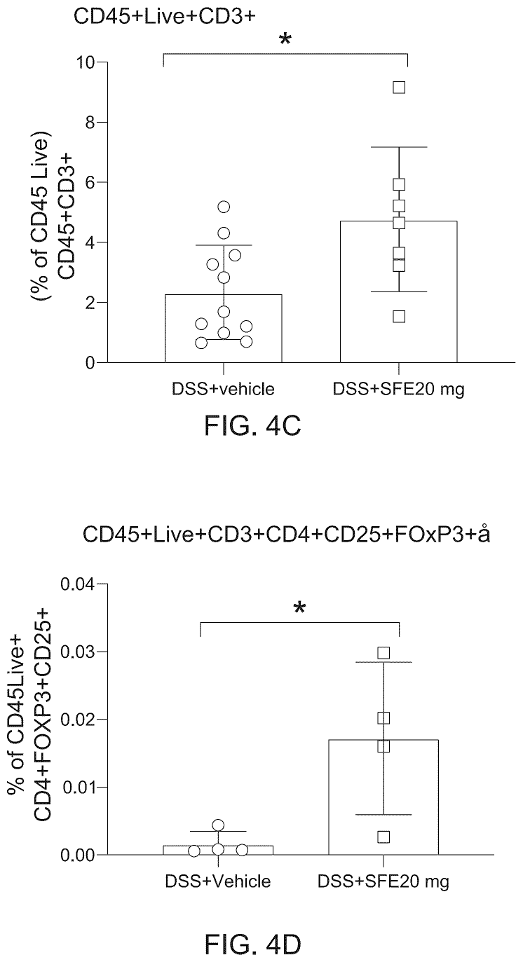

FACS analysis was performed on cells isolated from colon lamina propria of the DSS colitis mice treated with SFE (20 mg/kg) compared to vehicle control. SFE treatment decreased TNFα+ expressing pro-inflammatory macrophages (CD45+ Live+CD64+F4/80+ TNFα+) and increased CD206+ expressing anti-inflammatory macrophages (CD45 Live+CD64+F4/80+CD206+) (FIG. 4). Dendritic cells (DCs) also play an important role in IBD pathogenesis (Rutella, S. et al. “Intestinal dendritic cells in the pathogenesis of inflammatory bowel disease”, World journal of gastroenterology 2011; 17:3761-3775). and exist as several subtypes (Collin, M. et al. “Human dendritic cell subsets: an update”, Immunology 2018; 154:3-20). Furthermore, DCs are the most potent professional antigen presenting cells that prime T cells. Thus, it was sought to explore the effect of saffron treatment on total and IL-10 producing DCs. Suprisingly, a significant increase was observed in CD45 Live+CD64-CD11c+F480-DCs and this subset was enriched for IL10 expression by intracellular staining and FACS (FIG. 4A-B). Saffron treatment also increased CD45+CD3+ T cell population as well as FOXP3+ regulatory T cells (FIG. 4C, D).

### Saffron and its Constituent Safranal Decrease Secretion of Pro-Inflammatory Cytokines on T Cells in Human PBMCs

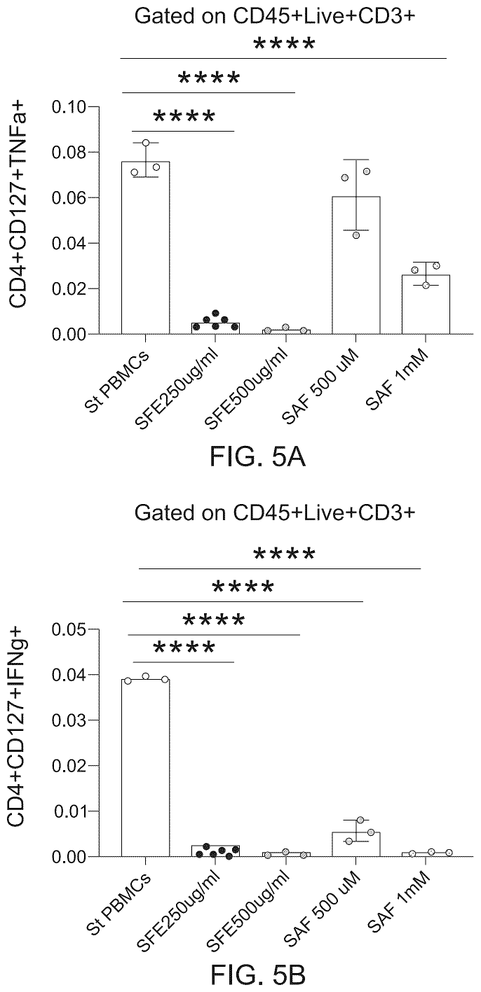

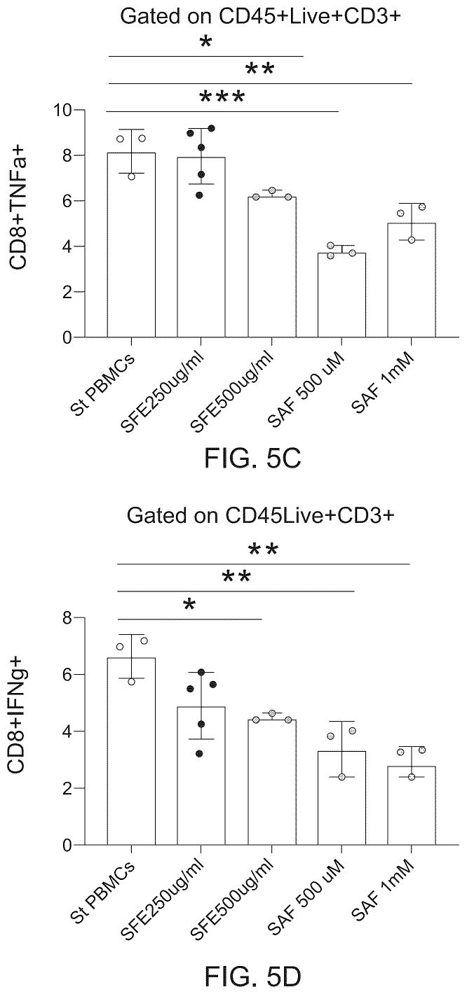

Flow cytometry data shows that SFE and Safranal decreases secretion of pro-inflammatory cytokines TNFα and IFN-γ on CD45+CD3+CD4+CD127+ (FIG. 5) and on CD45Live+CD3+CD45+CD3+CD8+ in comparison to media treated stimulated PBMC. The SFE doses 250 and 500 μg/ml and safranal doses (500 μM, 1 mM) showed inhibitory response towards TNFα+ and IFNg+ proinflammatory cytokines in CD4+ T cells. In CD8+ T cells, TNFα+ expression was reduced in presence of SFE dose 500 μg/ml and safranal doses (500 μM, 1 mM) only, while IFNg+ expression was reduced in all tested doses of SFE and safranal in comparison to stimulated human PBMCs treated with media.

### Saffron Treatment Induces Cytoprotective Transcription Factors and Downstream Anti-Inflammatory and Anti-Oxidant Proteins

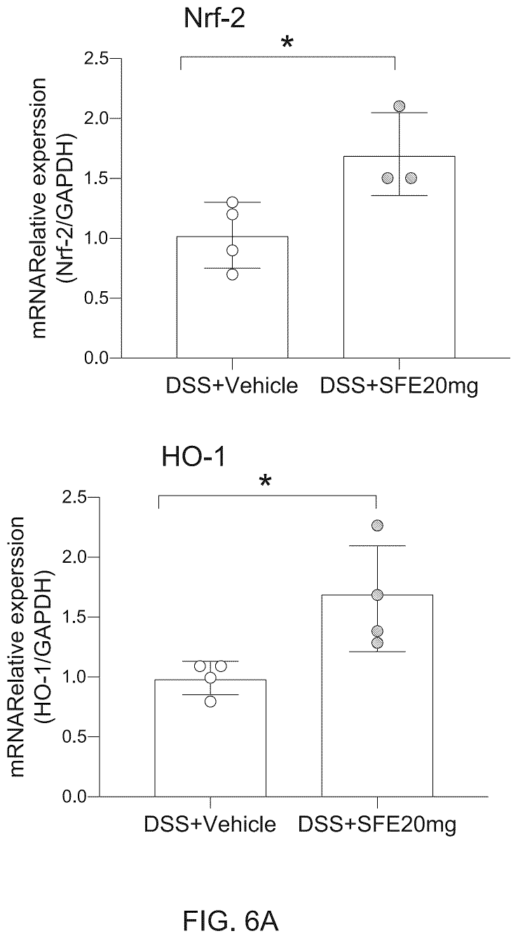

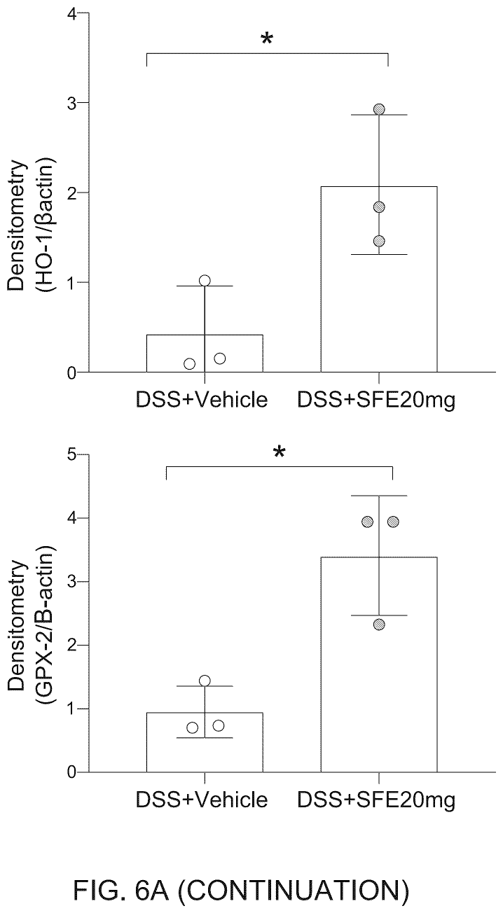

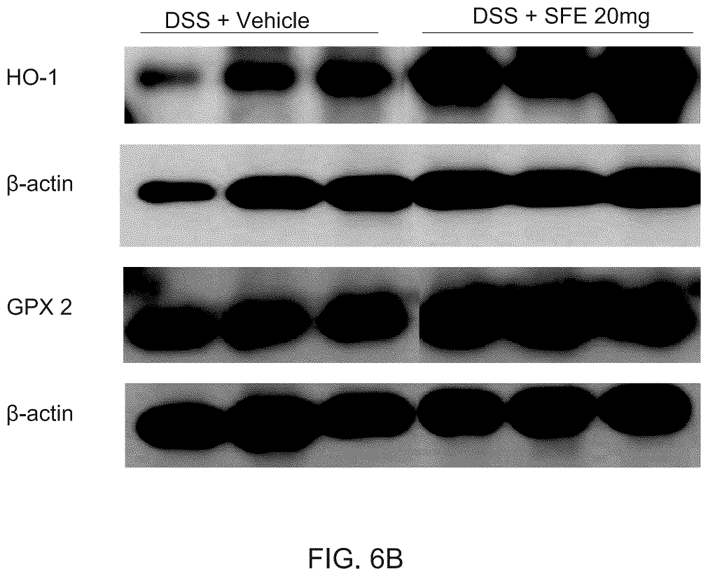

The effects of saffron treatment on the expression of NRF-2 and its downstream targets GPX-2 and HO-1 were analyzed. Saffron increased NRF-2 (n=3) and HO-1 (n=4) mRNA expression in DSS treated mice colons (FIG. 6A). Immunoblot analysis showed upregulation of HO-1 and GPX-2 proteins (downstream targets of NRF-2) expressions in the colon tissues (n=3), with saffron (DSS+ SFE20 mg) treatments as compared to control group (DSS+ Vehicle) mice (FIG. 6B).

Saffron Alters the Abundance of Microbial Taxa Associated with Inflammation

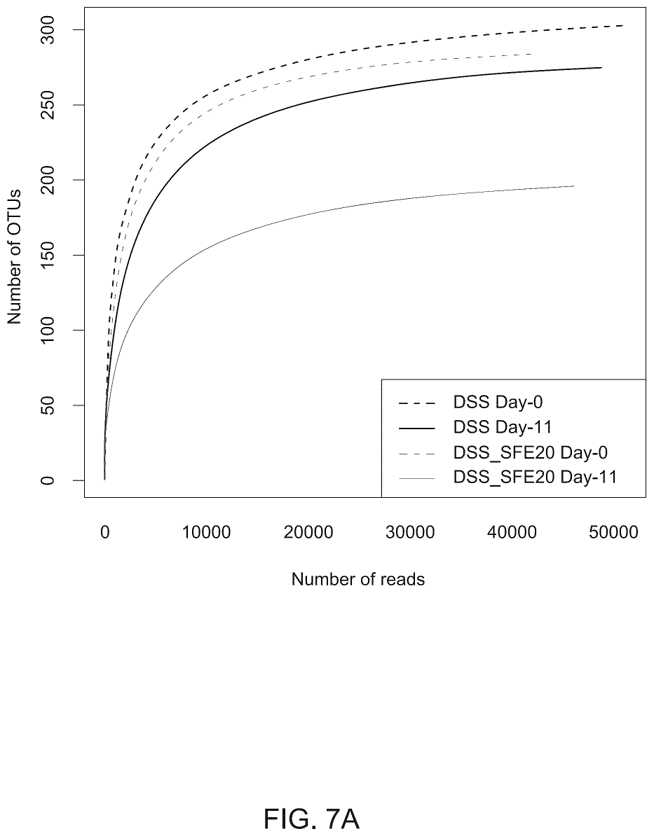

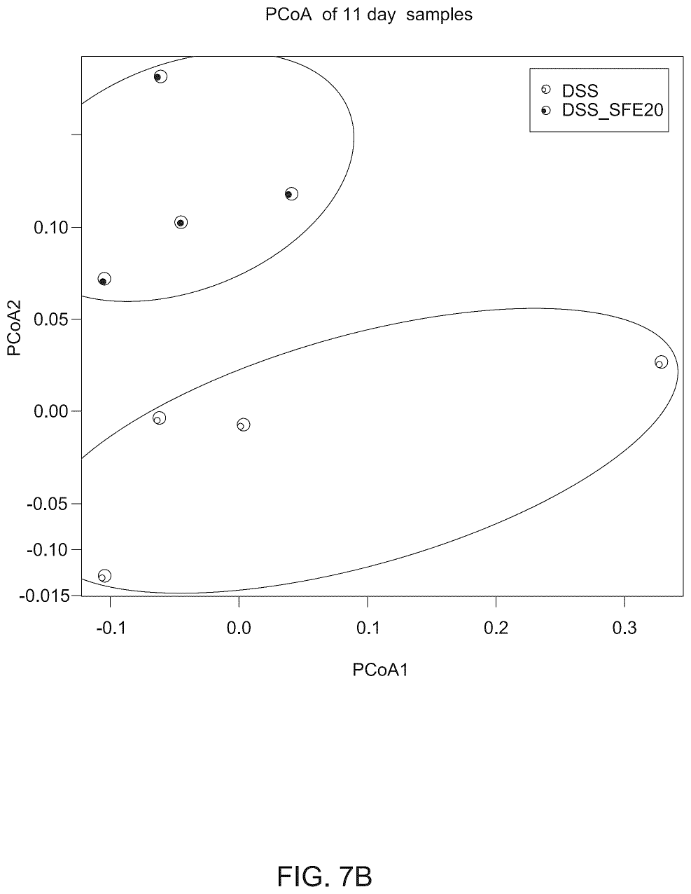

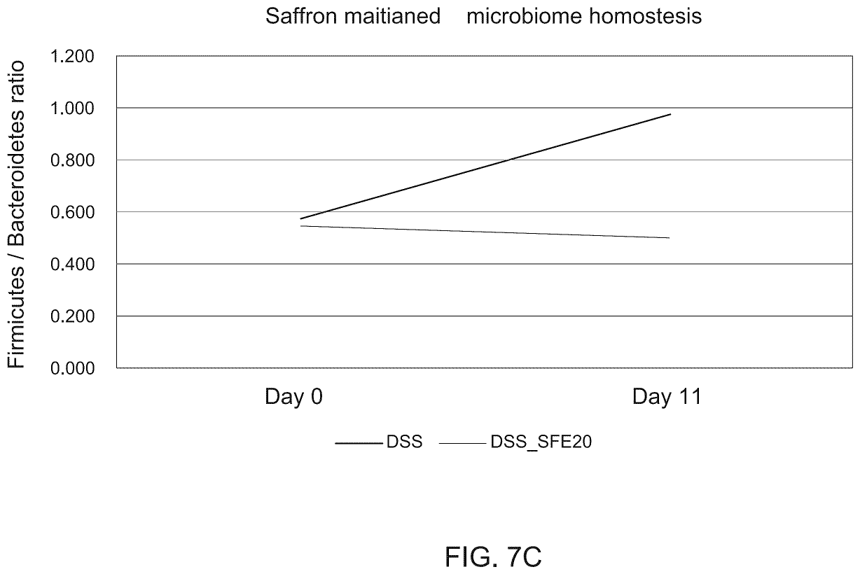

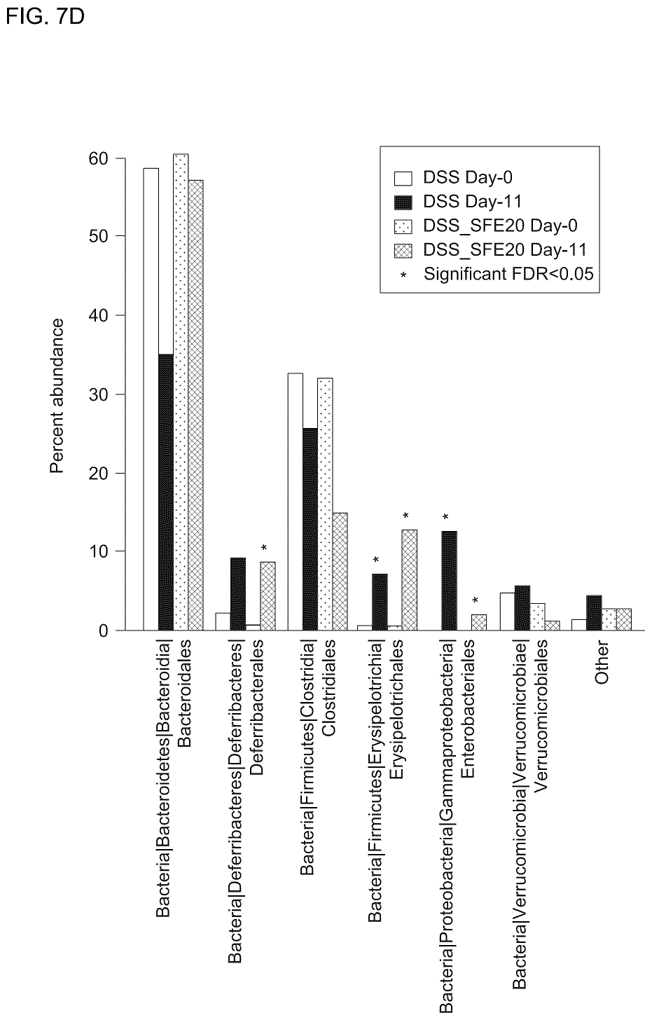

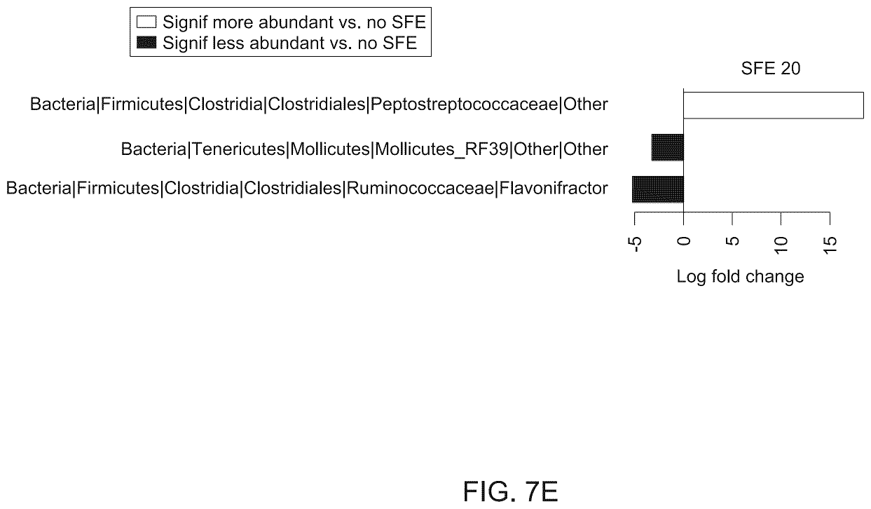

To determine the effects of saffron on the composition of the gut microbiota in the mice treated with DSS, a high-throughput sequencing of 16S rRNA was performed. Rarefaction and Shannon index analysis indicated that the sequencing depth covered the major and minor phylotypes and most of the diversity was accounted for. As shown in FIG. 7A, the average rarefaction curves for each experimental condition reveal that all the curves are close to saturation, indicating that the sequencing depth is adequate for the detection of all OTUs in the analyzed samples. Moreover, the saturation curve (curve flattening) at day 11 under saffron treatment was reached earlier than in the other experimental samples and at a lower number of sequencing reads, probably attesting to OTUs higher richness as a result of saffron 11 days treatment.

To further appreciate saffron-associated effect at the microbiota level, a principal coordinate analysis (PCoA) of the DSS and DSS-SFE samples was performed using UniFrac-based principal coordinate analysis (FIG. 7B). Color coded data from individual mice formed distinct components in this analysis. The ovals represent clustering by DSS treatment groups and were switched to those in the group along the PC1 axis in the saffron-treated group after the administration of saffron (FIG. 7B). This analysis further highlighted an effect of saffron on the gut microbiota composition, even in the presence of a potent dysbiotic agent such as DSS.

To define the nature of saffron effect on the microbiota in this colitis model, its impact on an important parameter in microbiome homeostasis that is the Firmicutes/Bacteroides ratio was assessed. This ratio was maintained constant between day 0 (0.579) and day 11 (0.501) in DSS treated mice gavaged with saffron. However, mice treated with DSS only had a high ratio at day 11 (0.974) compared to day 0 (0.579), attesting to an enrichment of Firmicutes and/or depletion of Bacteroides under DSS treatment (FIG. 7C). To further dissect saffron effect, its effect on the relative abundance of bacterial groups at the Order level (FIG. 7D) was assessed. This analysis showed differences at the Order level with some showing statistical significance. Indeed, bacteria from the Erysopelotrichales Order were significantly more abundant in saffron treated mice at day 11 when compared to DSS only treated mice at day 11. Bacteria of the Enterobacteriales and Verrucomicrobiales Orders were less abundant in saffron treated mice at day 11. The relative abundance of significantly altered mucosa-associated bacterial groups at the genus level were also analyzed. Two groups were significantly less abundant namely Mollicutes RF39 and Flavonifractor, while Peptostreptococcaceae bacteria were more abundant in saffron treated mice at day 11 (FIG. 7E). There was only one unspecified Peptostreptococcaceae genus out of 7 that was upregulated in saffron treated mice. However, the overall abundance of Peptostreptococcaceae increased in DSS and DSS SFE mice from 0 to 2773.5 and 1446.25, respectively, very likely as a result of DSS effect. It is noted that the increase in overall Peptostreptococcacea genera was more pronounced in DSS samples.

### SUMMARY

As shown above, the unfractionated saffron (aqueous extract) protects mice against experimental induced colitis by significantly decreasing pro-inflammatory macrophages (M1), while increasing anti-inflammatory (M2) macrophages, IL10+ dendritic cells (DCs) and overall IL10+ expression on CD45+ live leukocytes as well as CD4+ FOXP3+ regulatory T cells. Both innate and adaptive immune responses occurred through an activation of the NRF2, HO1, GPX2 pathway and a modulation of the gut microbiome that counter selects pro-inflammatory bacteria. The effects of unfractionated saffron (aqueous extract) in ameliorating experimental colitis in two mouse models were tested and the results showed that saffron has a protective role in ameliorating experimental colitis. In DSS model, it was found that SFE 20 mg/kg body weight treatment affected gross and histological features, evident by improved histology score, decreased DAI score, improved body weight and colon length and reduced fecal lipocalin levels.

To validate that these results are not model dependent, TNBS colitis model was used where SFE 20 mg/kg treatment also decreased colitis by improving colon length, body weight and decreasing fecal lipocalins levels. The above results showed that using unfractionated saffron (20 mg/kg) treatment showed significant reproducible protective effects in treating experimental colitis. The intestinal inflammation and lipocalin reduction results show that saffron attenuated colitis in pre-clinical models by alleviating inflammatory activities. The results showed that saffron is a potent modulator of both innate and adaptive immune responses. Indeed, anti-inflammatory T lymphocytes and DCs were more prominent in saffron treated mice. This finding was further confirmed in in vitro experiments where it was showed that saffron and its constituent safranal can also suppress secretion of pro-inflammatory cytokines on T cells in human PBMCs showing its potential translational significance in developing therapy for mild inflammation. It was showed that TNF-α and pro-inflammatory macrophages (M1) decreased. Anti-TNF-α drugs (i.e., infliximab, adalimumab, certolizumab pegol, golimumab) have been used in the last 20 years with good results for both CD and UC (Cote-Daigneault J, et al. “Biologics in inflammatory bowel disease: what are the data?” United European Gastroenterol J 2015; 3:419-28) with some side effects while saffron has not shown any adverse effect. It was also showed that TNFα+, IFNg+ on CD8+ T cells decreased in human PBMCs in vitro and in saffron and safranal treated animals which further confirm and define the anti-inflammatory properties of saffron. Saffron modulation of anti-inflammatory cytokines and growth factors (TNF-α, IFNγ, IL-1B, TGF-α, and platelet derived growth factors) may counter processes known to increase permeability through tight junctions disruption (Neurath, M. “Current and emerging therapeutic targets for IBD”, Nat Rev Gastroenterol Hepatol 2017; 14:688). Therefore, barrier dysfunction and inflammation that form in colitis can be blocked or attenuated by saffron.

Cytokines are considered crucial signals in the intestinal immune system. Immune cells, such as, T cells, dendritic cells, macrophages, and intestinal epithelial cells, are involved in the secretion of various cytokines that regulate the inflammatory response in UC (Luissint, A. C. et al. “Inflammation and the Intestinal Barrier: Leukocyte-Epithelial Cell Interactions, Cell Junction Remodeling, and Mucosal Repair”, Gastroenterology 2016; 151:616-32). Studies have revealed elevated levels of cytokines, such as TNF-α, IFN-γ, IL-1B, IL-6, IL-17, and IL-21, in UC (Luissint, A. C. et al. supra). The oral administration of saffron resulted in decreased levels of TNF-α, IFN-γ, and pro-inflammatory macrophages (M1;CD45+ Live +CD64+F4/80+ TNFα+) in colitis-induced mice (FIG. 4). Saffron also increased CD206+ expressing anti-inflammatory macrophages (CD45 Live+CD64+F4/80+CD206+; FIG. 3) along with dendritic cells (Ds subtypes). In addition, saffron treatment resulted in an increase in total and subset of IL-10 producing DCs (CD45 Live+CD64-CD11c+F480-DCs; FIG. 4A-B). IL-10 is known as a potent anti-inflammatory interleukin.

Saffron treatment also up-regulated CD45+CD3+ T cell population and their ability to initiate Foxp3+ Treg cell generation (FIG. 4C,D) and thus alleviated DSS-induced colitis. This is consistent with saffron protective role in the digestive tract with colitis dysregulated innate and adaptive immune responses. Since dendritic cells (DC) are the most important antigen presenting cells, and act as bridges connecting the adaptive and innate immune systems, saffron is considered to play a crucial role in the regulation of local homeostasis.

The potential mechanisms of saffron protective effects were further explored by investigating downstream anti-inflammatory effects that include activation of the NRF-2-HO-1 pathways. NRF-2 is a cytoprotective transcription factor that regulates cellular redox balance via the expression of genes encoding for anti-oxidant and anti-inflammatory proteins (Mitsuishi, Y. et al. “The Keap1-Nrf2 system in cancers: stress response and anabolic metabolism”, Front Oncol 2012; 2:200). Association of NRF-2 and HO-1 has been shown with crocin decreased cyclooxygenase-2 (COX-2), inducible nitric oxide synthase (iNOS), NF-κB activity, inflammatory cytokines such as TNF-α, in presence of saffron and its active ingredients, crocin (Kawabata, K. et al. supra; Khodir, A. E. et al. supra). It has been demonstrated that NRF2 and its target genes HO1 and GPX-2 are upregulated in the colons of mice treated with saffron showing its protective mechanism as anti-oxidant and anti-inflammatory.

It has been found that saffron induced a richness in the mice microbiota allowing all OTUs to be detected at a lower number of sequencing reads when compared to samples collected at day 0 with DSS and with DSS SFE. This applies to DSS day 11 samples as well. The PCoA further highlighted saffron effect on individual mice in each group. Indeed, the day 11 samples in DSS treatment clustered together and distinctly from the day 11 samples from DSS SFE 20 mg kg-1treatd mice. This finding led to pool samples from the mice in each treatment group for more specific analyses at the phylum, Order and Genus levels to establish saffron-induced differentials at the gut microbiota composition level.

At the phylum level, the two prominent gut microbial phyla in humans and mice are Firmicutes and Bacteroides. A stable ratio of these two phyla is a sign of a homeostatic gut microbiota. DSS treated mice had an increase in this ratio from day 0 to day 11 which might be due to an increase in Firmicutes and/or depletion of Bacteroides as a result of this colitis-inducing agent. Firmicutes were reported to be more prevalent in obesity and inflammation environments. This shift however was not noticed in the saffron treated mice which displayed similar Firmicutes/Bacteroides ratio at day 0 and day 11. This finding is surprising as the richness noted in the rarefaction analysis seems not to be random but more likely directed to counter DSS dysbiotic effect.

At the Order level, many bacterial Orders showed clear differences between DSS treated vs. DSS SFE treated mice. Three Orders were significant with bacteria from the Erysopelotrichales Order significantly more abundant in saffron treated mice at day 11 when compared to DSS only treated mice at day 11 while bacteria of the Enterobacteriales and Verrucomicrobiales Orders were less abundant in saffron treated mice at day 11. Schwab et al. have previously reported an increase of the low abundant Enterobacteriales, Deferribacterales, Verrucomicrobiales and Erysipelotrichales in DSS-induced colitis mice (Schwab, C. et al. “Longitudinal study of murine microbiota activity and interactions with the host during acute inflammation and recovery”, ISME J 2014; 8:1101-14). Three of these Orders were significantly reduced in abundance in the presence of saffron, which highlights saffron-microbiota modulating capacity to counter DSS dysbiotic effect. Only Erysopelotrichales Order showed a higher abundance in saffron treated mice when compared to DSS only treated mice. Dirk et al. showed that Crohn's disease status correlated strongly with a decreased abundance of Erysipelotrichales (Gevers, D. et al. “A Microbiome Foundation for the Study of Crohn's Disease”, Cell Host Microbe 2017; 21:301-304) consistent with the results presented herein.

At the genus level, the anti-inflammatory effect of saffron through a modulation of the gut microbiota was further confirmed. Indeed, two genera showed significant decrease in abundance in the presence of saffron, namely Flavonifractor and Mollicutes. These two genera have previously been shown to associate with strong pro-inflammatory responses. For example, Flavonifractor was reported as associating with strong pro Inflammatory markers (C5a, IL-6, IL-8, IL-7, IL-1B, IL17A and IL-21) and saffron inhibition of Flavonifractor can be associated with its anti-inflammatory effect (Huang, S. et al. “The imbalance of gut microbiota and its correlation with plasma inflammatory cytokines in pemphigus vulgaris patients”, Scand J Immunol 2019; 90: e12799; Silwedel, C. et al. “More than just inflammation: Ureaplasma species induce apoptosis in human brain microvascular endothelial cells”, J Neuroinflammation 2019; 16:38). The downregulation of these two well established inflammatory bacteria links directly saffron effect to immune response modulation. However, the genus that was found more abundant in saffron treated mice that is Peptostreptococcaceae was reported as associating with colitis. This finding led to further explore all detected Peptostreptococcacaea in the samples which led to 7 genera that cumulatively were shown to be more abundant in DSS treated mice than in DSS SFE mice. This finding on its own highlights the anti-colitis effect of saffron since even in the presence of DSS, the increase of bacteria from this genus was slowed and kept at half the abundance obtained with DSS only (highlighting a potential countereffect in saffron treated mice that displayed half level abundance from 2773.5 vs. 1446.25, Table 1). In sum, saffron treatment at the genus level led to a significant reduction of three pro-inflammatory bacterial genera, one of which is Enterobacteriales that is known to contain many pathogens that exacerbate inflammation at the gut (Mao, G. et al. “Depolymerized RG-I-enriched pectin from citrus segment membranes modulates gut microbiota, increases SCFA production, and promotes the growth of Bifidobacterium spp., Lactobacillus spp. and Faecalibaculum spp”, Food Funct 2019; 10:7828-7843). These findings indicate that saffron anti-inflammatory effects are vehiculated through a modulation of the gut microbiota where pro-inflammatory bacteria are counter selected even in the presence of potent colitis-inducing agents such as DSS (Litvak, Y. et al. “Dysbiotic Proteobacteria expansion: a microbial signature of epithelial dysfunction”, Curr Opin Microbiol 2017; 39:1-6).

TABEL 1
Relative abondance of Peptostreptococcaceae in DSS and DSS saffron treated mice

| | DSS | DSS | [symbol] SS_SFE20 | DSS_SFE20 |
|---|---|---|---|---|
| | Day-0 | Day-11 | Day [symbol] | Day-11 |
| Bacteria\|Firmicutes\|Clostridia\|Clostridiales\|Peptostreptococcaceae\|Romboutsia | 0 | 1382 | 0 | 876.75 |
| Bacteria\|Firmicutes\|Clostridia\|Clostridiales\|Peptostreptococcaceae\|Romboutsia | 0 | 1347.25 | 0 | 436.75 |
| Bacteria\|Firmicutes\|Clostridia\|Clostridiales\|Peptostreptococcaceae\|Terrisporobacter | 0 | 0 | 0 | 5.25 |
| Bacteria\|Firmicutes\|Clostridia\|Clostridiales\|Peptostreptococcaceae\|Other | 0 | 0 | 0 | 113 |
| Bacteria\|Firmicutes\|Clostridia\|Clostridiales\|Peptostreptococcaceae\|Romboutsia | 0 | 37.75 | 0 | 0 |
| Bacteria\|Firmicutes\|Clostridia\|Clostridiales\|Peptostreptococcaceae\|Paeniclostridium | 0 | 6.5 | 0 | 8.5 |
| Bacteria\|Firmicutes\|Clostridia\|Clostridiales\|Peptostreptococcaceae\|Paraclostridium | 0 | 0 | 0 | 6 |

[symbol]

indicates data missing or illegible when filed

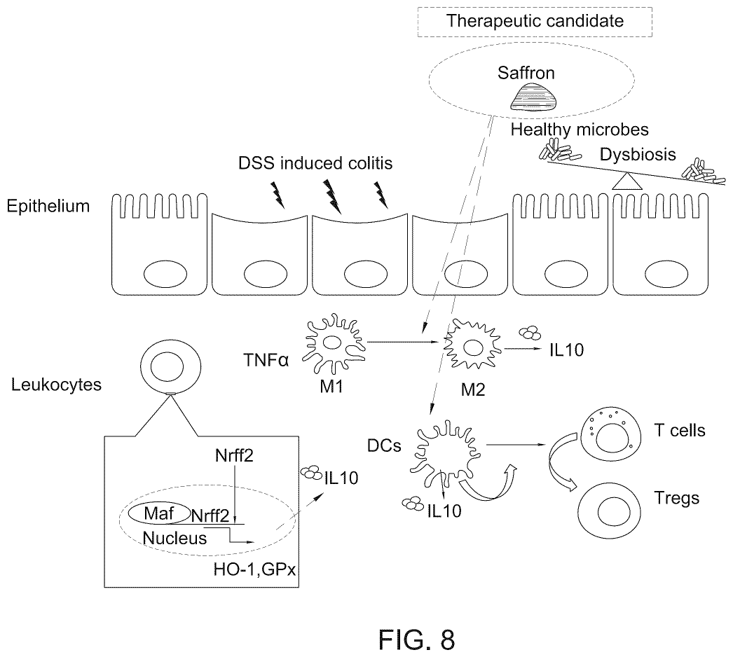

In conclusion, saffron given orally can reduce intestinal inflammation in colitis mice models. Saffron plays an important role in the modulation of the immune response and downstream anti-inflammatory signals involving cytoprotective antioxidant NRF-2 and HO-1 pathways (FIG. 8). Taken together, protective role of saffron was shown and potential action mechanisms was uncovered using independent acute colitis models. Safe therapeutic options are offered to treat mild clinical IBD (or added in combination treatments) across different age groups with inclusion of at-risk individuals such as the elderly.

### Clinical Trial

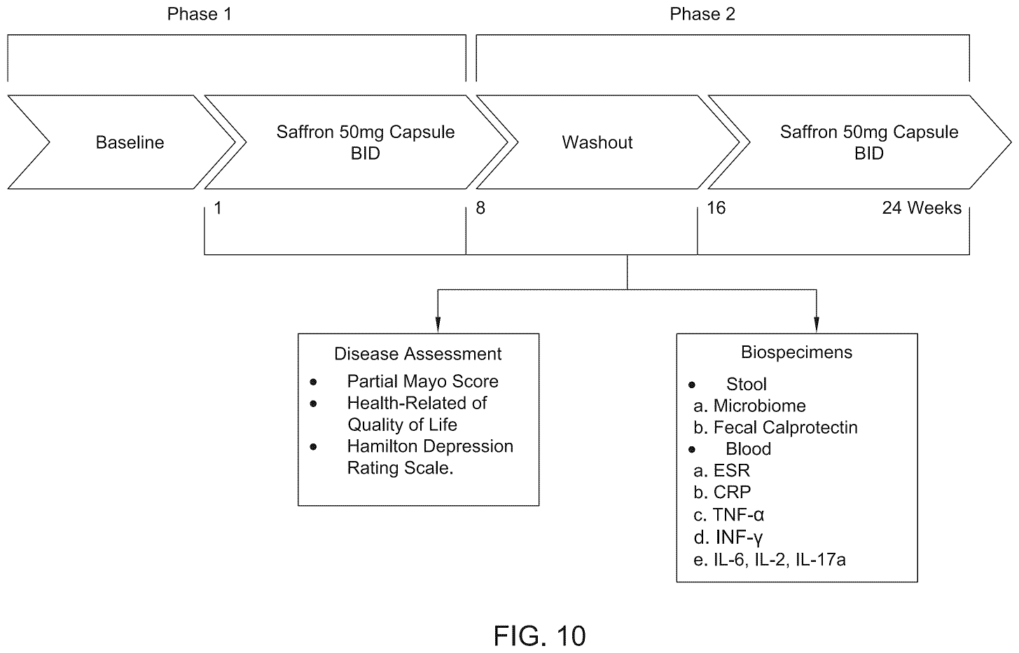

Four patients with a disease history of mild to moderate UC of at least three months and a confirmed UC diagnosis by colonoscopy were asked to provide written informed consent and to be compliant with the schedule of protocol assessments, treatment plans, laboratory tests, and other study procedures. Baseline laboratory tests were performed for serum immunomarkers, CRP, ESR, Fecal-Calprotectin, and stool microbiome and metabolome. The clinical disease activity was measured using a Partial Mayo Score (Lewis, J. D. et al., “Use of the noninvasive components of the Mayo score to assess clinical response in ulcerative colitis”, Inflammatory bowel diseases, 2008. 14 (12): p. 1660-1666; the content of which is incorporated herein by reference). Later, patients were given Saffron 50 mg capsule twice daily as an intervention for 8-weeks, followed by an 8-week washout period, and then given Saffron 50 mg twice daily for additional 8-weeks. Laboratory tests along with clinical disease activity were measured again at the end of 8-weeks post-treatment with Saffron (Phase 2) (FIG. 10).

Only three (3) patients completed all requirements. Patient 2 (P2) dropped out of the trial. The baseline demographics and characteristics of the three patients are listed in Table 2 below.

TABLE 2

| Characteristics | Patient 1 (P1) | Patient 3 (P3) | Patient 4 (P4) |
|---|---|---|---|
| Age (years) | 49 | 62 | 25 |
| Sex | Male | Female | Female |
| Race | African American | African American | White |
| Duration of UC | 14 | 8 | 9 |
| diagnosis (years) | | | |
| Location of UC | Proctosigmoiditis | — | Pancolitis |
| BMI | 33.6 | 33.8 | 22.3 |
| Smoking Status | Never | Ex-smoker | Never |
| Level of Education | Post- graduate | Post- graduate | Post- graduate |
| Medications | Mesalamine | Mesalamine | Mesalamine |
| Prominent Diet (Veg/meat) | Meat | Meat | Both |
| Presenting symptoms | Abdominal Pain, | None | Abdominal Pain, |
| | Diarrhea | | Diarrhea, Rectal |
| | | | Bleed occasionally |

### Data and Sample Collection

Patients completed a comprehensive demographic and medical history questionnaire at baseline. Study assessments were performed during four visits: baseline (visit 1), post-treatment (visit 2), after washout (visit 3), and Phase 2-post-treatment with saffron (visit 4). Patients were instructed to fast before each visit, and data collection was identical for all visits. At visits 1, 2, 3 and 4, a partial Mayo Score, Hamilton Depression Rating Scale (HDRS), blood and stool samples were collected.

### Sample and PBMN Isolation

Patients were requested to provide blood samples at baseline pre-treatment with Saffron and 1-3 days after 8-weeks of treatment with Saffron. Blood samples were collected to measure serum CRP and PBMN isolation was done according to the method for measuring the inflammatory markers such as IL-2, IL-6, IL-17a, TNFα, INFγ, ESR and CRP, disclosed in Singh, G. et al., “Protective Effect of Saffron in Mouse Colitis Models Through Immune Modulation”, Digestive Diseases and Sciences, 2021: p. 1-14, the content of which is incorporated herein by reference. Specifically, both blood with and without Heparin centrifuged at 2000 rpm for 10 minutes were used. Red top tubes were used for serum (top layer) collection, and the remainder of the blood was discarded. Blood for plasma and PBMC in green top tubes contain Heparin to prevent the blood from clotting (Catalog: 366486 BD). Plasma was collected (top layer) and stored (pure, without freezing media) in cyrotube.

Remaining blood was transferred into a 50 mL conical tube, and diluted 1:1 in PBS without Ca²⁺/Mg²⁺. In a 50 mL Accuspin tube (Cat: A2055, Sigma-Aldrich), 15 mL of Ficoll (Cat: 14-1440-03, GE Healthcare) was poured and centrifuged at 2000 rpm for 30 seconds to spin the Ficoll down. The diluted blood was gently layered on top of the Accuspin filter, without breaking the ficoll layer. The sample was layered over Ficoll and centrifuged for 20 minutes at 2000 rpm at 21° C., with acceleration at 5 and break at 0 (these settings are very important). Using a serological pipette the buffy coat PBMC layer was collected and transferred into a new 50 mL tube. The new buffy coat tube was filled to 50 mL with PBS and spinned at 2000 rpm for 10 mins. The supernatant was aspirated from the pellet, then the pellet was resuspended in 1 mL PBS and then filled to 50 mL. The tube was centrifuged again at 2000 rpm for 10 minutes. The supernatant was aspirated from the pellet and the pellet was resuspended in chilled PBS. For a small pellet, 1 mL PBS was used. The cells were counted by hemocytometer by adding 10 μL cell suspension to 90 μL blue stain, and mixed. The cells spin and aspirated supernatant, then the cells were concentrated by being suspended in Recovery Media (Cat. 12648010, ThermoFischer). The cells ˜3.5 million cells/mL (or 5 million and 10 million cells/mL tubes) were aliquoted into each cryotube and labelled and placed in the cryotubes pre-chilled to 4° C. and then in the −80° C. freezer. Cryotubes were moved to liquid N₂ after 24 hr.

### Stool Sample

Each individual immediately provided a fresh stool sample, and delivered to the Howard research center on an ice bag. The patients with UC provided an additional stool sample 1-3 days post-treatment with Saffron. Any sample remaining at room temperature for more than two hours was discarded, and stool samples stayed at 80° C. for future use. These stool samples were used to measure fecal calprotectin level, which is a marker for intestinal inflammation. Stool samples were also used to identify the targeted microbiome at baseline and post-treatment with Saffron.

### Immunomarker Analysis by CyTOF

CyTOF was used for immune markers IL-2, IL-6, IL-10, IL-17A, TNFα, and INFγ. PBMCs were characterized with respect to TNF-α, IFN-γ, IL-6, IL-17, IL-2, IL-4 and IL-10 phenotypes in patients treated with low and high dose saffron, using CyTOF according to Subrahmanyam, P. B. et al., “Mass Cytometry Analysis of T-Helper Cells”, in T-Helper Cells. 2021, Springer. p. 49-63, the content of which is incorporated herein by reference.

### Analysis

Targeted immune marker analysis was done in PBMCs based on CyTOF preliminary clinical pilot data and Brim, H. et al., “Microbiome analysis of stool samples from African Americans with colon polyps”, PLOS One, 2013. 8 (12): p. e81352, the content of which is incorporated herein by reference. The analysis was restricted to markers of specific importance for UC such as TNF-α, IFN-γ, IL-6, IL-17, IL-2, IL-4 and IL-10. State-of-the-art methods such as sample barcoding and normalization beads were used, to ensure reliable data with minimal batch effects.

### Gut Microbiome

DNA extraction and 16S rDNA analysis: DNA was extracted from the stool samples using QIAamp DNA Stool Mini kits according to the manufacturer's instructions (Qiagen, Germantown MD, US) and completed based on Brim, H. et al., Supra. Specifically, DNA quality was assessed using Nanodrop 2000 and gel electrophoresis. All samples yielded good quality and good amounts of DNA for bacterial community analysis.

For 16S rDNA analysis, a PCR amplification was performed prior to Next Generation Sequencing as described in Brim, H. et al., Supra, and Brim, H. et al., “A Microbiomic Analysis in African Americans with Colonic Lesions Reveals Streptococcus sp. VT162 as a Marker of Neoplastic Transformation”, Genes (Basel), 2017. 8 (11), the content of which is incorporated herein by reference. Specifically, DNA extracts were amplified using primers that targeted the 16S rRNA genes. These primers included adaptor sequences as well as unique 12 bp barcodes incorporated onto the reverse primer such that each sample had a unique barcode. Using approximately 100 ng of extracted DNA, the amplicons were generated with Platinum Taq polymerase (Invitrogen, CA, USA) using the following cycling conditions: 95° C. for 5 min for an initial denaturing step followed by 35 cycles of: 95° C. for 30 s, 55° C. for 30 s, 72° C. for 30 s, followed by a final extension step of 72° C. for 7 min, and then stored at 4° C. PCR amplicons were purified using the QIAquick PCR purification kit (Qiagen Valencia, CA, USA), quantified, normalized, and then pooled in preparation for sequencing using an Illumina HiSeq platform according to the manufacturer's protocol (Illumina Inc., San Diego, CA).

Details of OTU assembly from reads, abundance estimation of each OTU and taxonomic assignment are as described in Subrahmanyam, P. B. et al., Supra, and Brim, H. et al., Genes (Basel), 2017. 8 (11), supra. OTU abundances were loaded into R (version 3.5.1) using the package described in McMurdie, P. J. et al., “phyloseq: an R package for reproducible interactive analysis and graphics of microbiome census data”, PLOS One, 2013. 8 (4): p. e61217, the content of which is incorporated herein by reference. Rarefaction curves were calculated using the R package vegan. The R package DESeQ2, described in Love, M. I. et al, “Moderated estimation of fold change and dispersion for RNA-seq data with DESeq2”, Genome Biol, 2014. 15 (12), p. 550, the content of which is incorporated herein by reference, was used to identify taxonomic units that were significantly differentially abundant within the analyzed groups. At each taxonomic rank, the read counts falling into OTUs within each taxon were summed. These counts were then used for differential abundance analysis assuming a negative binomial distribution. The threshold for statistical significance was a false detection ratio (FDR)<0.05. For Firmicutes/Bacteroidetes ratios, the total counts in the two phyla were calculated and the ratio of the counts for each sample was computed. For differential analysis of ratios, the Wilcoxon Rank Sum test was used on the ratios for each group (CTRL vs. saffron and baseline and post treatment).

### Results

### Dietary Supplement Saffron Decreased Systemic Inflammation

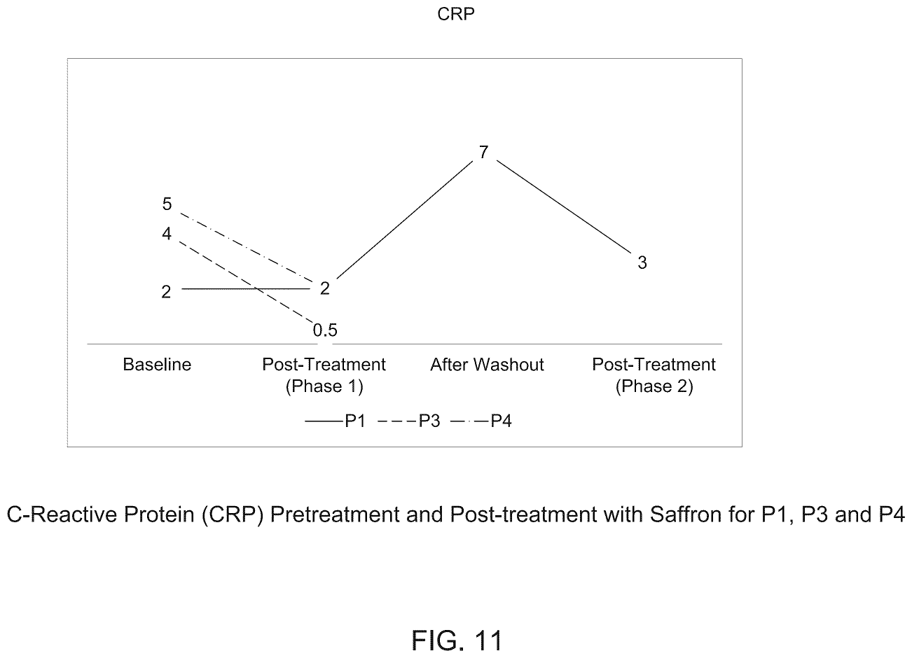

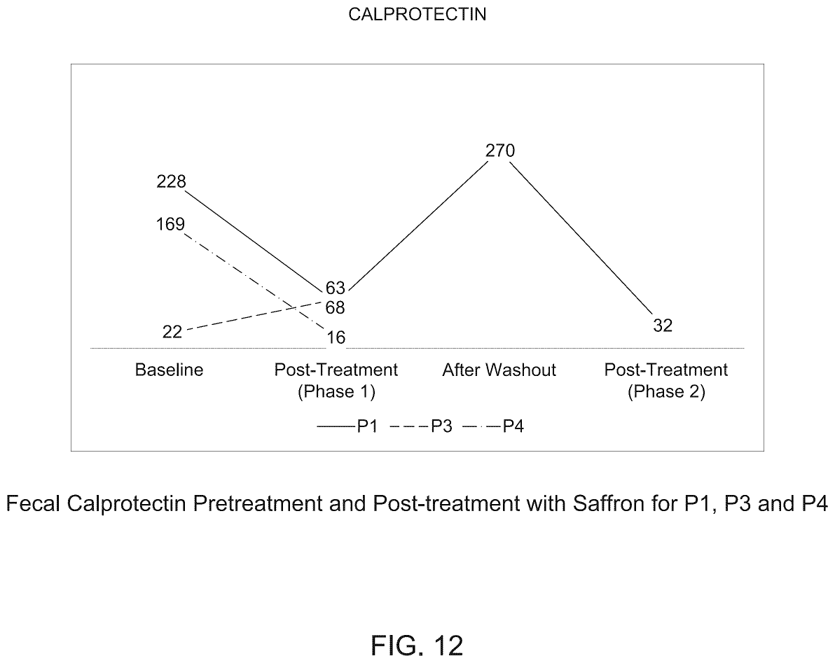

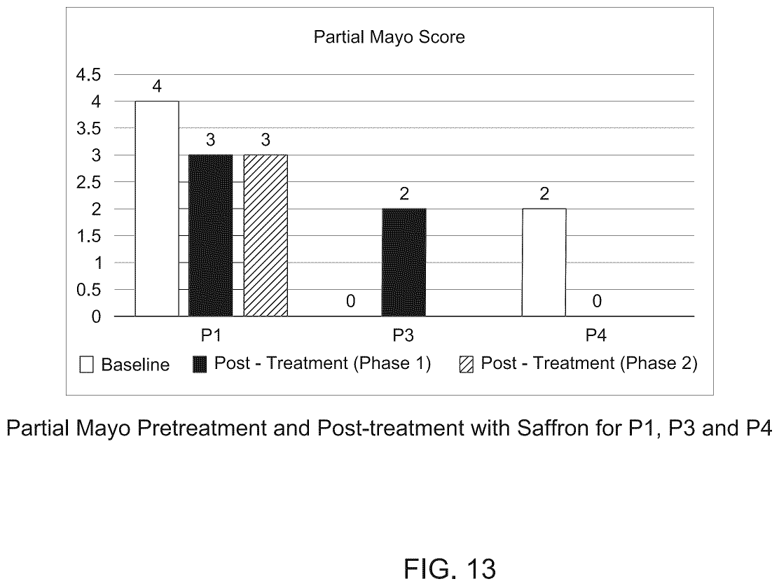

The serum inflammatory markers ESR and CRP were under normal levels at baseline in P1, P3, and P4. Compared to the baseline, there was a decrease in systemic inflammatory markers such as ESR, CRP and calprotectin in P1, P3 and P4 (FIGS. 11 and 12). The stool calprotectin levels were reduced markedly in P1 and P4, but P3 showed increased fecal calprotectin levels post-treatment with Saffron for 8-weeks. During Phase 2, Fecal Calprotectin, a stool inflammatory marker levels increased to 270 μg/g post washout of saffron for about 8-weeks. Again, saffron treatment for the next 8-weeks after the washout showed a reduction in calprotectin levels to 32 μg/g (FIG. 13).

### Daily Intake of Saffron Improved UC Disease Activity and Clinical Response in Patients 1 and 4

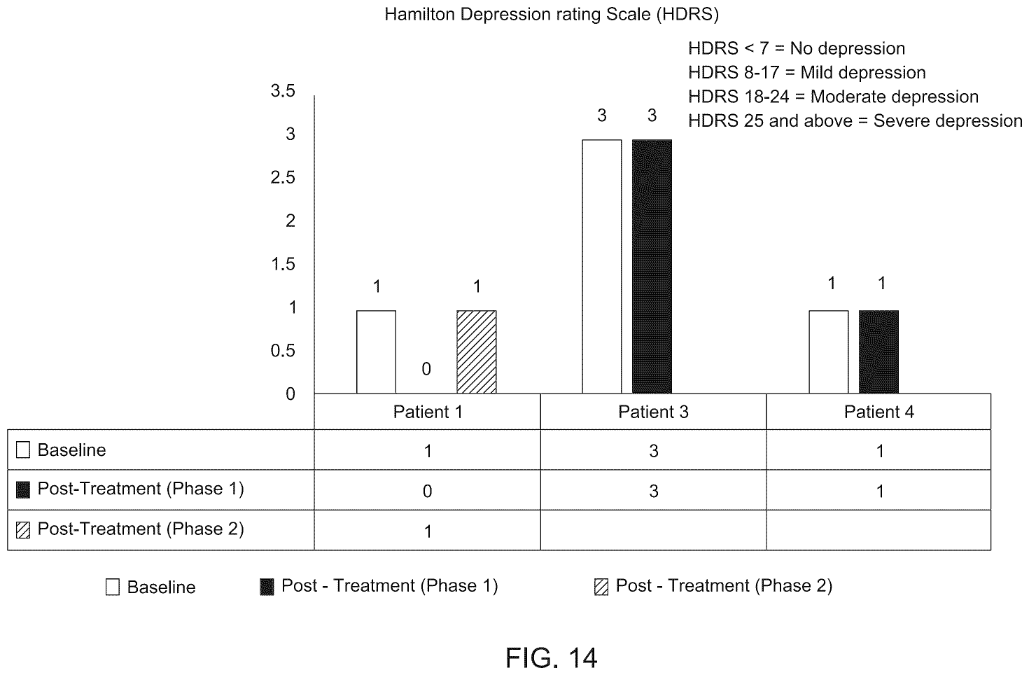

The health-related quality of life, measured through the HRQOL questionnaire, showed remarkable improvement post-treatment with Saffron for 8-weeks in P1 and P4 from baseline by improvement in presenting symptoms such as severity of abdominal pain, rectal bleeding, and diarrhea. Improvement was observed in the clinical disease activity by Partial Mayo Score post-treatment with 50 mg of Saffron twice daily for 8-weeks in patients 1 (P1) and 4 (P4) (FIG. 13). However, patient 3 (P3) showed worsening of partial mayo scores. After a washout period post-treatment with saffron again for 8-weeks in P1 the Partial Mayo Score remained the same. However, Hamilton Depression Rating Scale (HDRS) showed mixed results such as improvement in patient 1, no change observed in P3, whereas P4 showed deterioration on HDRS post-treatment with Saffron (FIG. 14).

### Immunomarker Measure by CyTOF

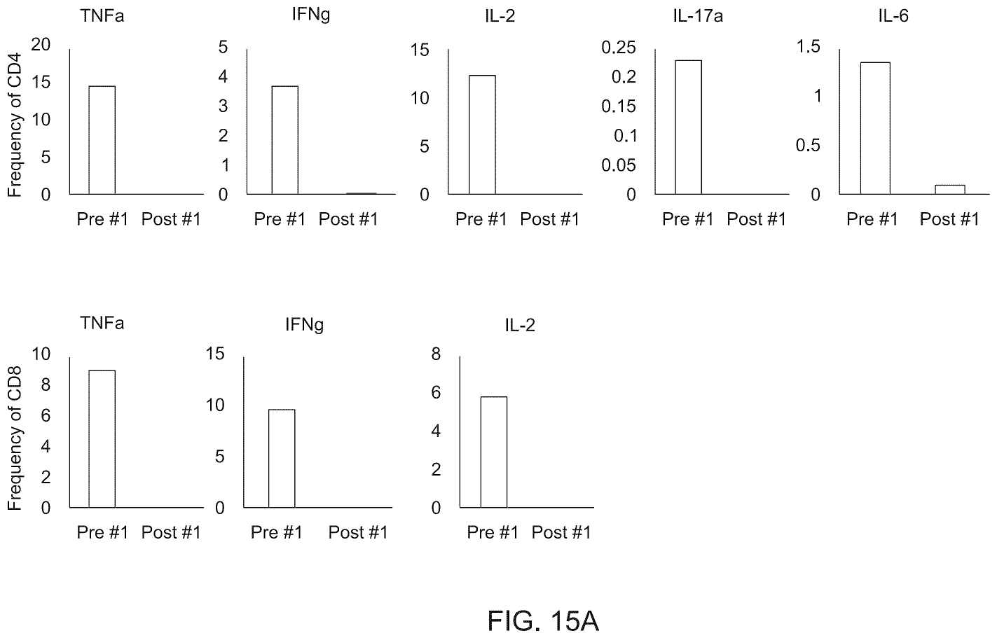

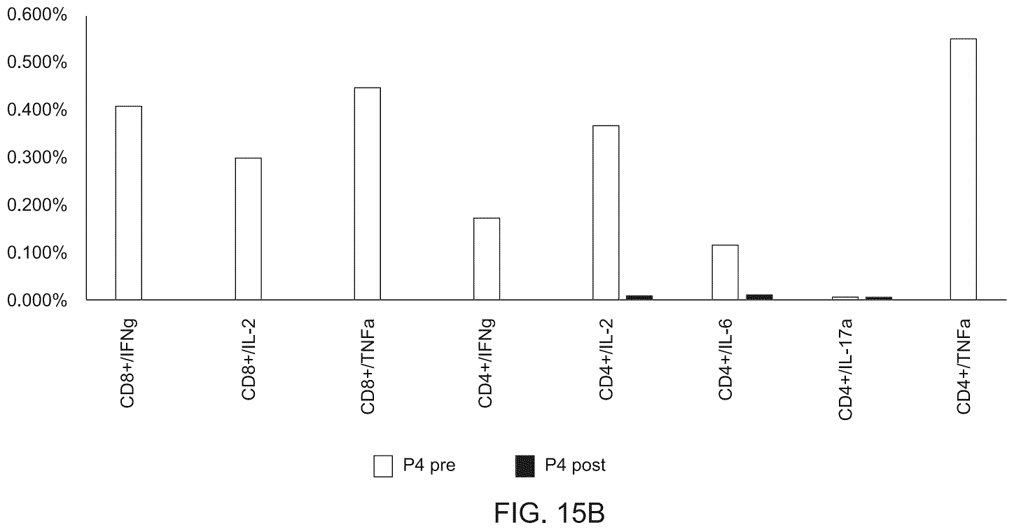

With 38 T cell and other lineage markers, including cell-surface phenotyping and intracellular staining, the CyTOF ICS assay was ideal for comprehensively measuring PBMCs subsets and their functions. The results showed that most of the pro-inflammatory markers to be reduced while anti-inflammatory markers are induced by saffron, as per the preclinical and clinical pilot data presented herein. 8-weeks of Saffron treatment in this UC patient decreased IL-2, IL-6, IL-17a, INF-y, and TNF-α cytokine production from CD4+ T cells and IL-2, IFN-g, and TNF-α production from CD8+ T cells in response to PMA/ionomycin stimulation, compared to initial pre-treatment baseline values (FIGS. 15A and 15B).

### Effects of Saffron on Stool Microbiome of Ulcerative Colitis Patients

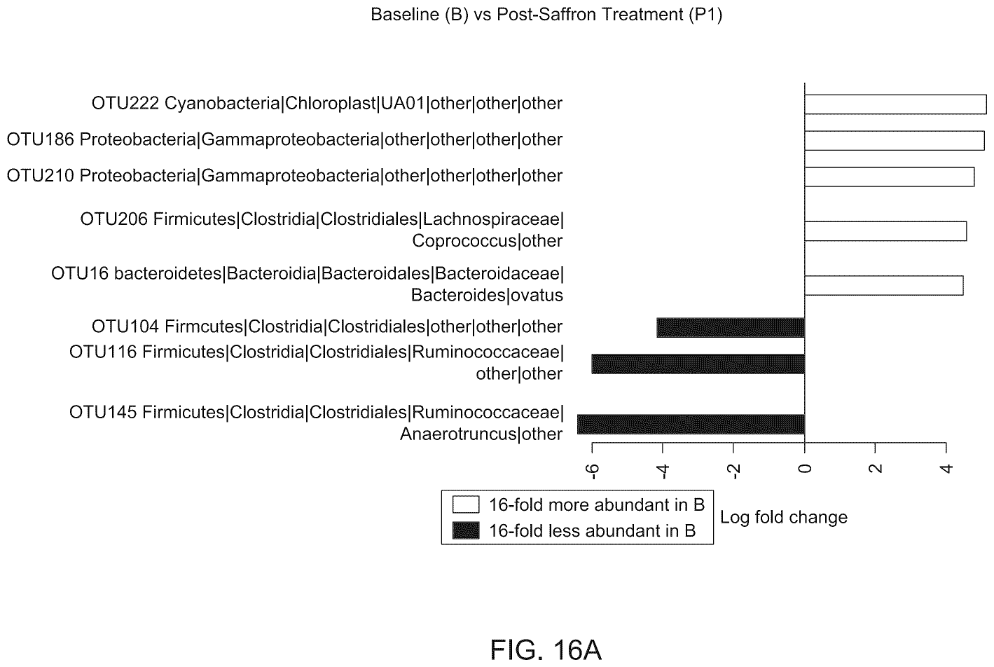

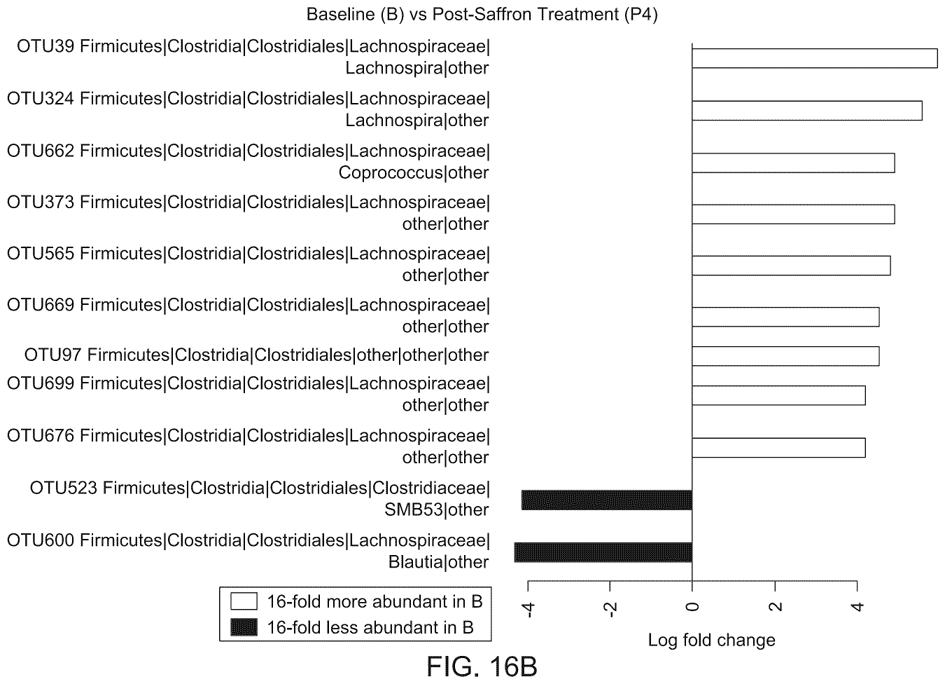

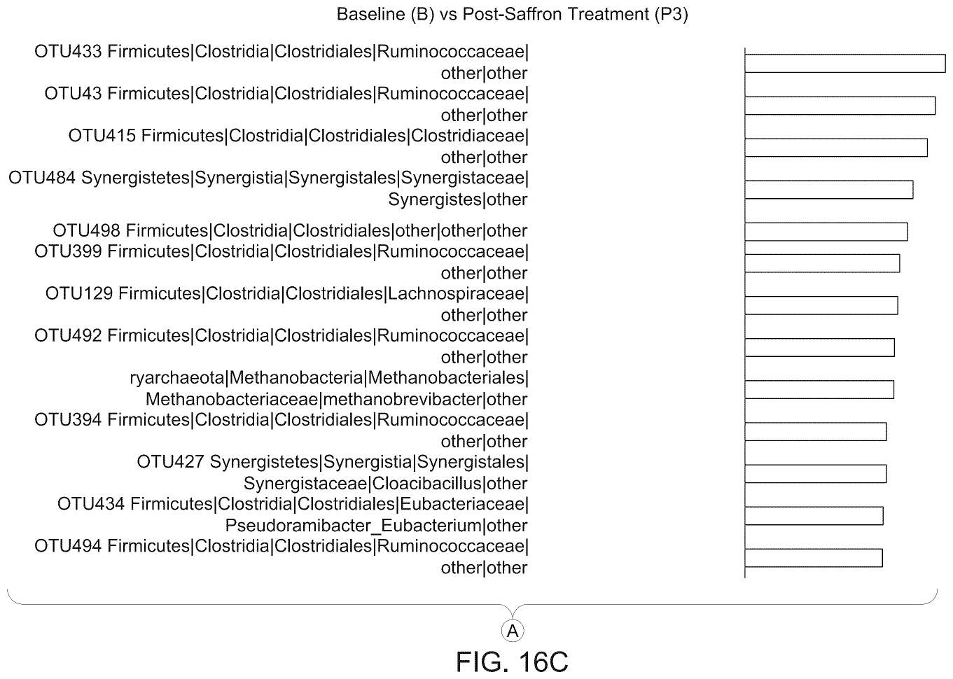

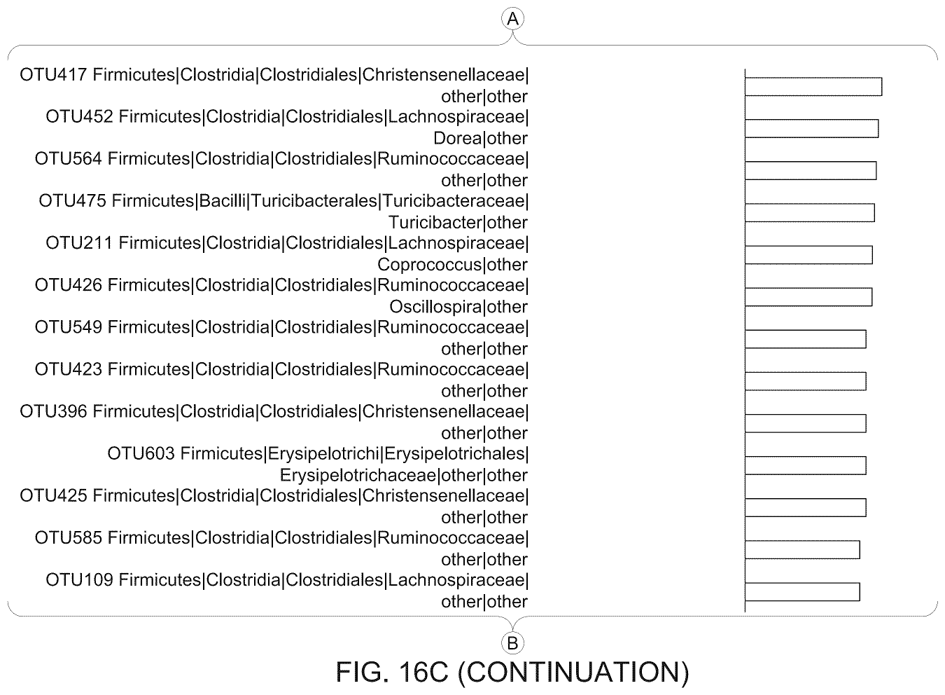

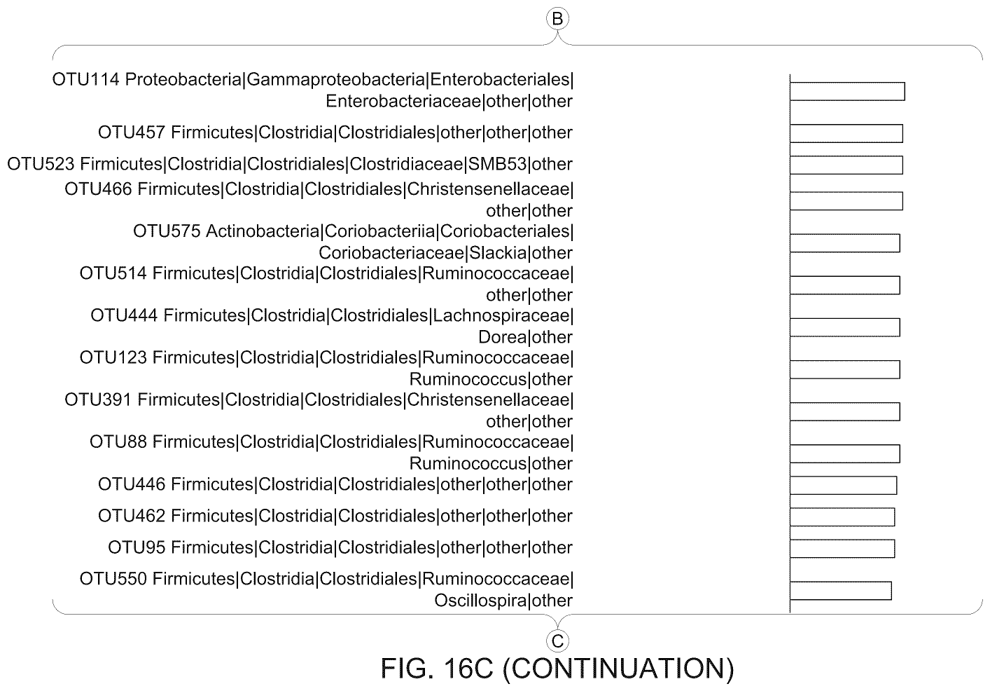

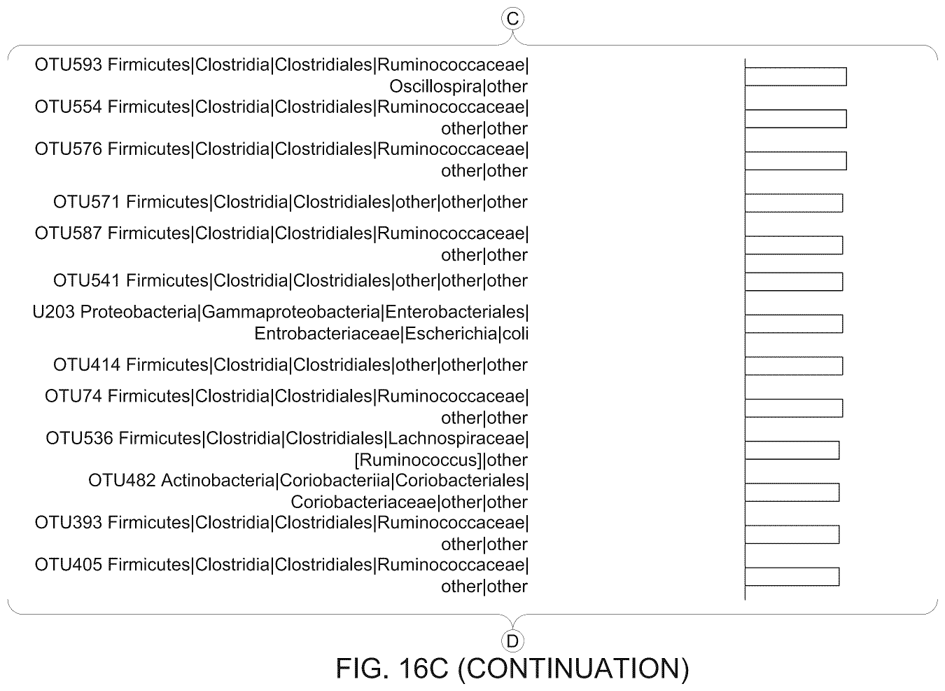

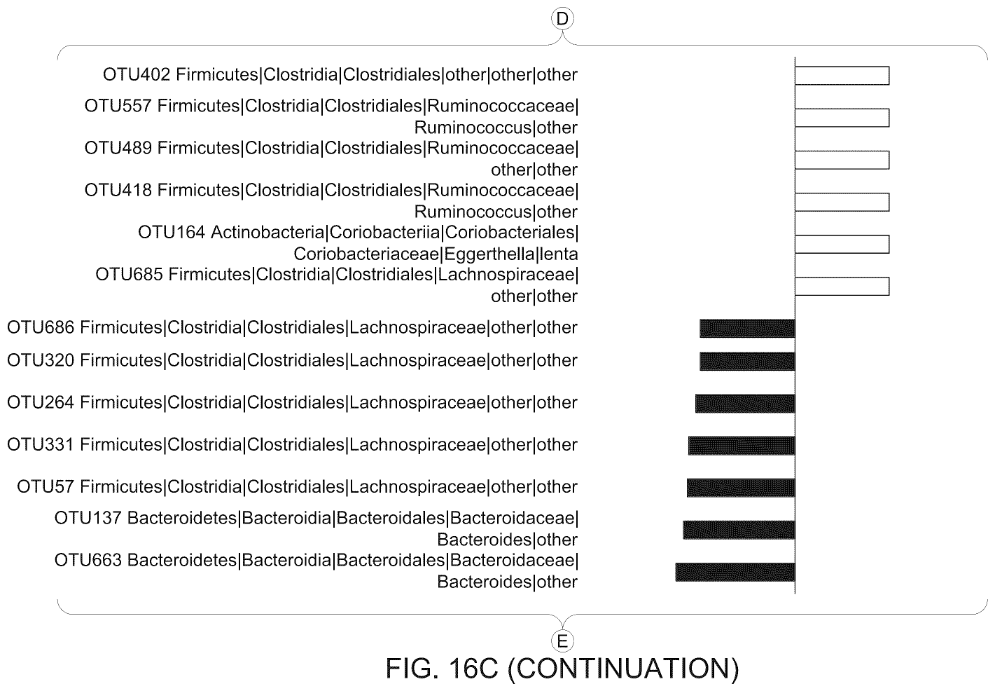

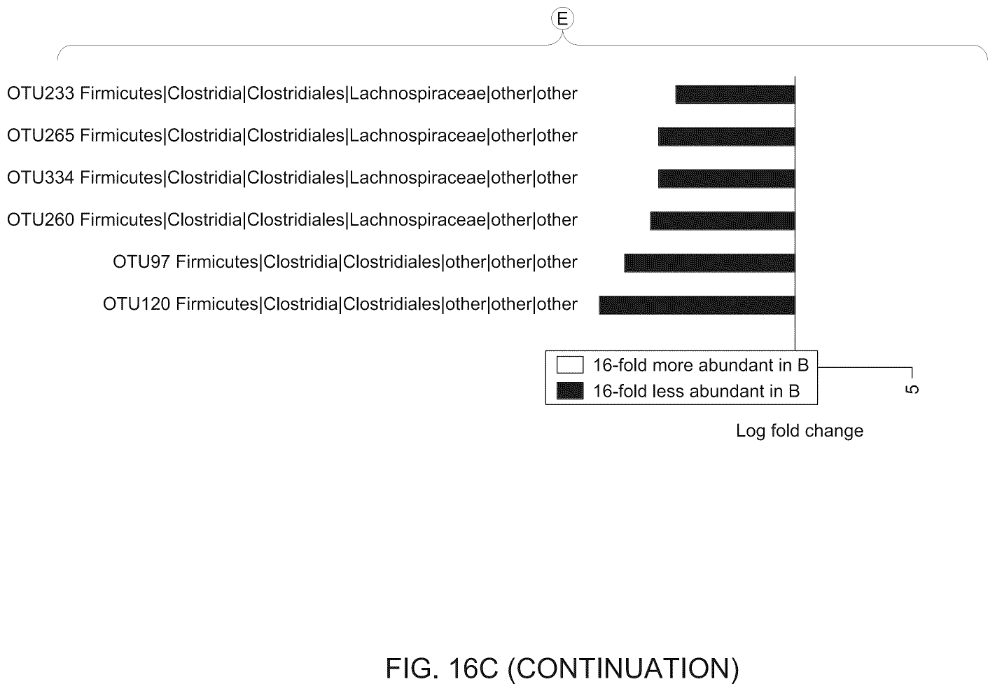

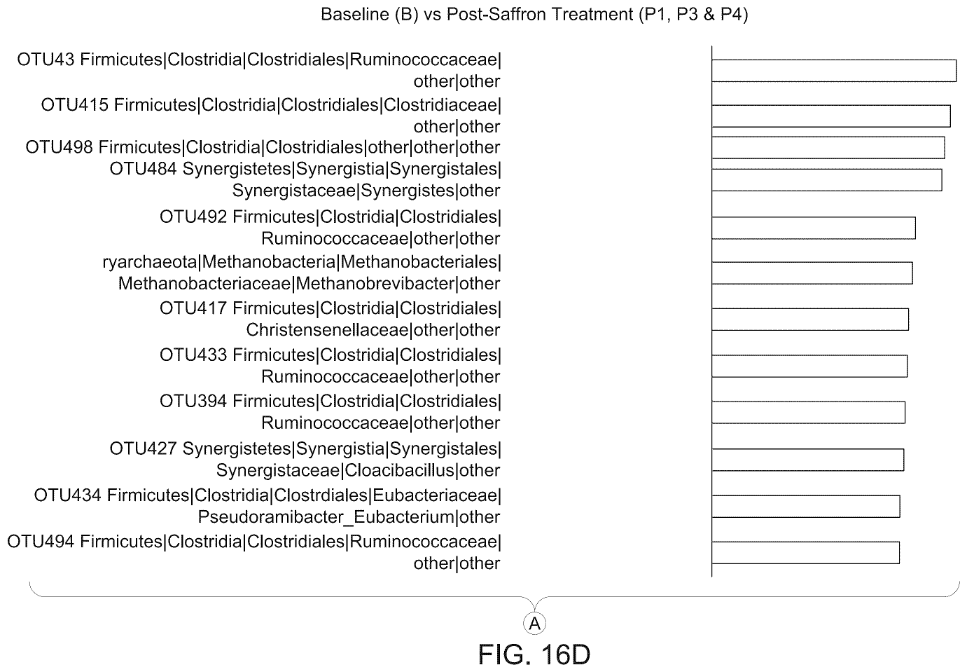

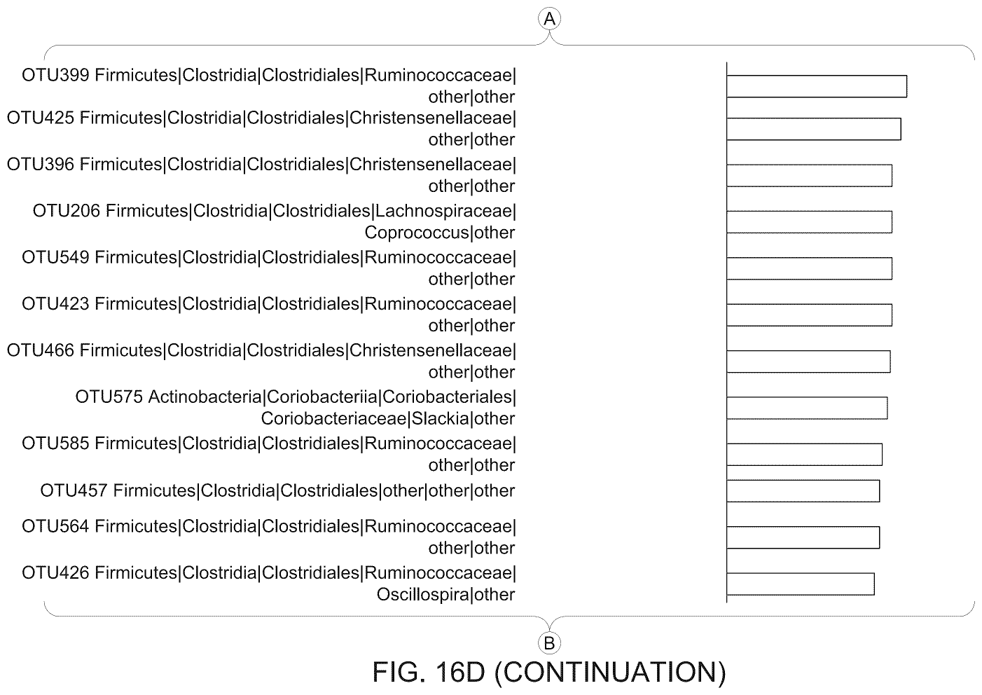

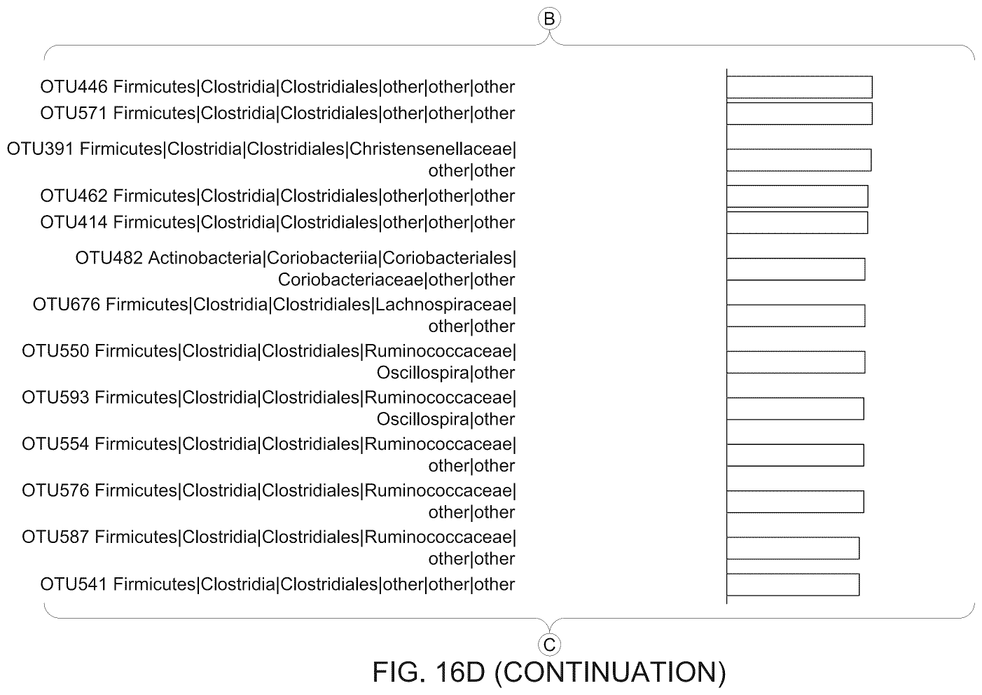

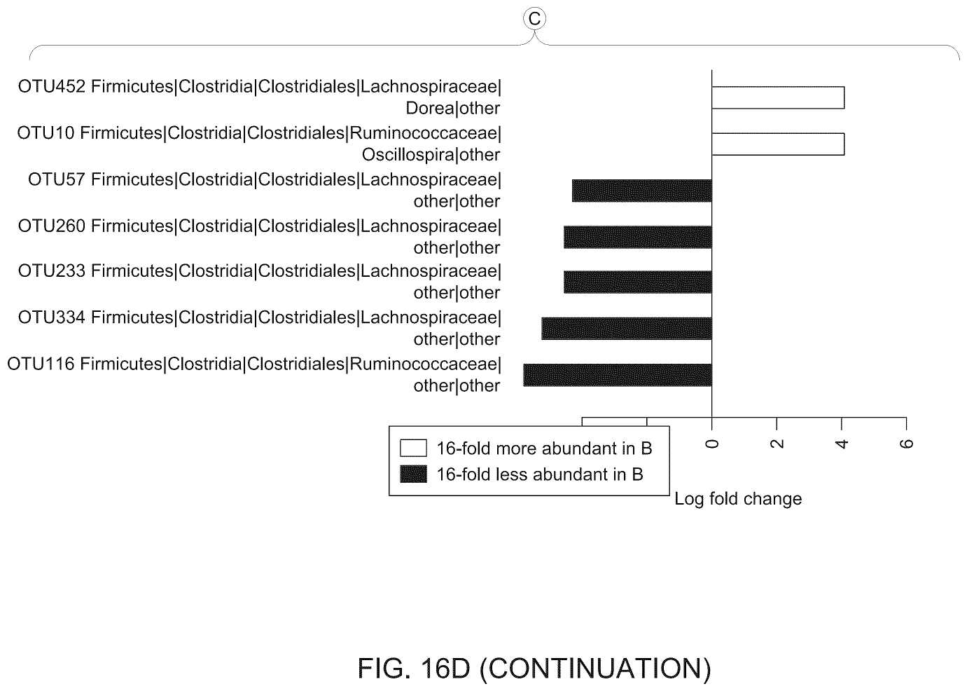

To determine whether saffron alters the human gut microbiota composition, the stool microbiome were analyzed at baseline and post-treatment with saffron for 8-weeks. Treatment with saffron was directed to significant changes at the phylum level of the stool microbiome. A dramatic reduction of Cyanobacteria, Proteobacteria, and Bacteroidetes in P1 and a less dramatic decrease in Firmicutes, Actinobacteria phyla in P1, P3 and P4 were identified post-treatment with saffron for 8-weeks (FIGS. 16A-16D).

The above results showed that saffron supplementation for 8-weeks among UC patients improved the anti-inflammatory status and reduced disease severity. Saffron led to significant improvements in clinical symptoms and HRQOL, decreased CRP and Calprotectin compared with baseline. Abdominal pain, diarrhea, and rectal bleeding presenting complaints at enrollment for P1 and P4 improved post-treatment with Saffron. A difference in disease severity and clinical response in UC patients' post-treatment with Saffron through Partial Mayo Score was successfully identified. Clinical response is defined as a decrease of at least 2 points of the Mayo Clinical Score from baseline (Turner, D. et al., “A systematic prospective comparison of noninvasive disease activity indices in ulcerative colitis”, Clinical Gastroenterology and Hepatology “, 2009. 7 (10): p. 1081-1088; the content of which is incorporated herein by reference).

Calprotectin (CP) is an established clinical biomarker for inflammatory bowel diseases and harbors immune-regulatory functions. Fecal CP concentrations found in healthy individuals mainly range between ˜10-50 μg/g stool (Jukic, A. et al., “Calprotectin: from biomarker to biological function”, Gut, 2021). As discussed above, the CP levels significantly decreased in the P1 and P4 post-treatment with Saffron. In P1, the CP levels, which decreased to normal post-treatment during Phase 1, immediately raised to 270 μg/g post-washout during phase 2. This suggests that Saffron has a significant role in reducing intestinal inflammation.

The above results confirm that after usage of Saffron, Saffron could ameliorate colitis and colitis-related colon carcinogenesis induced chemically in animals by decreasing mRNA expression of some pro-inflammatory cytokines and inducible inflammatory enzymes including IL-1B, IL-6, TNF-α, INF-γ, NF-κB, iNOS and COX-2 synthase in the colorectal mucosa and by increasing in the nuclear factor erythroid 2-related factor 2 (Nrf2) mRNA expression of the mice that received DSS (Singh, G. Supra; Kawabata, K. et al. supra). The pro-inflammatory cytokine production such as IL-6, IFN-γ, TNF-α, IL-2 from CD8+ T cells and CD 4+ T cells is much lower in the patients treated with 8-weeks of Saffron compared to baseline.

Intestinal microbiome dysbiosis, delineated by an increase in the number of mucosa-associated bacteria and a decrease in the overall biodiversity, has been consistently described in patients with IBD (Palmela, C. et al., “Adherent-invasive Escherichia coli in inflammatory bowel disease”, Gut, 2018. 67 (3): p. 574-587). Specific microbial genera, such as Lactobacillus, Bifidobacterium, and Faecalibacterium, may hinder the inflammatory response in the intestine by decreasing the expression of pro-inflammatory cytokines and inciting the production of anti-inflammatory cytokines. Enterobacteriaceae, a large class of gram-negative facultative bacteria, are commonly linked to many inflammatory diseases like IBD. In addition, the depletion of the phyla Firmicutes and the increase of the Proteobacteria populations have been linked to human IBD (Lobionda, S. et al., “The role of gut microbiota in intestinal inflammation with respect to diet and extrinsic stressors”, Microorganisms, 2019. 7 (8): p. 271). The above results showed a decrease in Proteobacteria, Actinobacteria, and some genera of Firmicutes phylum at the end of 8-weeks of treatment with Saffron.

In conclusion, Saffron is an effective treatment for patients with active mild to moderate UC. Administration of Saffron resulted in reduced disease activity, improved clinical response, and reduced serum levels of CRP, fecal calprotectin, and pro-inflammatory cytokines after eight weeks of treatment. Therefore, using saffron supplements, along with conventional medicines, may have beneficial impacts on UC patients.

While the subject matter disclosed herein has been described in connection with what is presently considered to be practical example embodiments, it is to be understood that the present disclosure is not limited to the disclosed embodiments, and covers various modifications and equivalent arrangements included within the spirit and scope of the present invention.

What is claims is:

1. An agent for treatment or mitigation of inflammatory bowel disease (IBD), comprising saffron as an active ingredient.

2. The agent according to claim 1, wherein saffron is in the form of saffron extract.

3. The agent according to claim 1, wherein the IBD is ulcerative colitis.

4. A method for treating or mitigating inflammatory bowel disease (IBD), comprising administering an effective amount of saffron to a subject in need thereof.

5. The method according to claim 4, wherein saffron is administered orally.

6. The method according to claim 4, wherein saffron is in a liquid or solid formulation.

7. The method according to claim 4, wherein saffron is in the form of saffron extract.

8. The method according to claim 4, wherein crocin, safranal and picrocrocin work synergistically as anti-inflammatory compounds.

9. The method according to claim 4, wherein the IBD is ulcerative colitis.

10. The method according to claim 4, wherein saffron treatment improves gross and histological features.

11. The method according to claim 4, wherein saffron decreases fecal lipocalin.

12. The method according to claim 4, wherein saffron treatment alters innate immune cell profiles.

13. The method according to claim 4, wherein saffron and its constituent safranal decrease secretion of pro-inflammatory cytokines on T cells in human PBMCs.

14. The method according to claim 4, wherein saffron treatment induces cytoprotective transcription factors and downstream anti-inflammatory and anti-oxidant proteins.

15. The method according to claim 4, wherein saffron alters the abundance of microbial taxa associated with inflammation.

16. The method according to claim 4, wherein saffron is administered from 5 mg to 100 mg per dose.

17. The method according to claim 16, wherein saffron is administered once or twice per day.
